(19) United States
(12) Patent Application Publication
(10) Pub. No.: US 2025/0161220 A1
(43) Pub. Date: May 22, 2025

(54) LYMPHATIC DELIVERY OF NANOPARTICLES TO TREAT NEURODEGENERATIVE, NEUROLOGICAL, AND EYE CONDITIONS

(71) Applicant: The Cleveland Clinic Foundation, Cleveland, OH (US)

(72) Inventor: Vinod D. LABHASETWAR, Cleveland, OH (US)

(21) Appl. No.: 18/839,121

(22) PCT Filed: Feb. 16, 2023
(86) PCT No.: PCT/US2023/062725

§ 371 Date: Aug. 16, 2024

Related U.S. Application Data
(60) Provisional application No. 63/310,723, filed on Feb. 16, 2022.

(51) Int. Cl. A61K 9/19; A61K 9/16; A61K 31/222; A61K 38/44; A61K 45/06

(52) U.S. Cl. CPC A61K 9/19; A61K 9/1623; A61K 9/1635; A61K 9/1647; A61K 9/1658; A61K 31/222; A61K 38/44; A61K 38/446; A61K 45/06; C12Y 111/01006; C12Y 115/01001

(57) ABSTRACT

Provided herein are composition, systems, kits, and methods for preventing and/or treating, reversing, and/or inhibiting progression of, a neurodegenerative and/or neurological conditions and/or eye conditions (e.g., retinitis pigmentosa) by administering a plurality of nanoparticles to a subject via subcutaneous, intramuscular, or intraperitoneal injection such that the plurality of nanoparticles are taken up by lymphatic capillaries and are transported to neurological and/or eye tissue of the subject via the subject's lymphatic system, wherein the plurality of nanoparticles encapsulate, or are attached or absorbed to, at least one drug agent.

The present application claims priority to U.S. Provisional application Ser. No. 63/310,723, filed Feb. 16, 2022, which is herein incorporated by reference in its entirety.

This invention was made with government support under W81XWH-16-1-0786 awarded by the Department of Defense and NS092033 and EY026340 by the National Institutes of Health. The government has certain rights in the invention.”

### FIELD

Provided herein are composition, systems, kits, and methods for preventing and/or treating, reversing, and/or inhibiting progression of, a neurodegenerative and/or neurological conditions and/or eye conditions (e.g., retinitis pigmentosa) by administering a plurality of nanoparticles (e.g., 3-30 nm) to a subject via subcutaneous, intramuscular, or intraperitoneal injection such that the plurality of nanoparticles are taken up by lymphatic capillaries and are transported to neurological and/or eye tissue of the subject via the subject's lymphatic system, wherein the plurality of nanoparticles encapsulate, or are attached or absorbed to, at least one drug agent.

### BACKGROUND

Oxidative stress is implicated in the onset and progression of Alzheimer's Disease (Aβ) by promoting multiple post-translational modifications of proteins, oxidative DNA damage, and lipid peroxidation. The oxidative damage accelerates the expression of beta-secretase-1 (BACE1), an enzyme involved in Amyloid-β (Aβ) generation. In turn, increased Aβ leads to increased oxidative stress and mitochondrial dysfunction. This vicious feed-forward cycle is repeated, causing progressive neurodegeneration in AD. Therefore, targeting oxidative stress is considered central to breaking the self-replicating vicious cycle that leads to progressive neurodegeneration. Similarly, oxidative stress contributes to the pathogenesis of Retinitis Pigmentosa (RP), which results in photoreceptor degeneration and is a major cause of visual impairment and blindness. The retina is particularly sensitive to oxidative damage because of its high oxygen demand, its high content of unsaturated lipids, and its exposure to light.

### SUMMARY OF THE INVENTION

Provided herein are composition, systems, kits, and methods for preventing and/or treating, reversing, and/or inhibiting progression of, a neurodegenerative and/or neurological conditions and/or eye conditions (e.g., retinitis pigmentosa) by administering a plurality of nanoparticles to a subject via subcutaneous, intramuscular, or intraperitoneal injection such that the plurality of nanoparticles are taken up by lymphatic capillaries and are transported to neurological and/or eye tissue of the subject via the subject's lymphatic system, wherein the plurality of nanoparticles encapsulate, or are attached or absorbed to, at least one drug agent.

In some embodiments, provided herein are methods of treating a neurodegenerative condition and/or neurological condition and/or an eye condition with nanoparticles comprising: administering a composition comprising a plurality of nanoparticles to a subject via subcutaneous, intraperitoneal, or intramuscular injection such that the plurality of nanoparticles are taken up by lymphatic capillaries of the subject and are transported to neurological tissue and/or eye tissue of the subject via the subject's lymphatic system, wherein the nanoparticles optionally have an average diameter of about 10-115 nm (e.g., 10 . . . 20 . . . 40 . . . 50 . . . 70 . . . 85 . . . 90 . . . 100 nm . . . 115 nm) or about 20-100 nm, or about 60-90 nm when measured in a dry state such as by transmission electron microscopy (TEM), or 30 to <350 nm (e.g., 30 . . . 50 . . . 70 . . . 110 . . . 140 . . . 170 . . . 210 . . . 260 . . . or 340 nm) when measured in the hydrated state using dynamic light scattering, commonly referred to as hydrodynamic diameter; wherein the subject has a neurodegenerative condition and/or neurological condition and/or eye condition and wherein the plurality of nanoparticles encapsulate, or are attached or absorbed to, at least one drug agent. In certain embodiments, the nanoparticles: i) have an average diameter of about 10-115 nm, about 20-100 nm, or about 60-90 nm as measured by TEM, or a hydrodynamic diameter of 30 to 340 nm, or about 200-250 nm, and/or ii) wherein said nanoparticles optionally have a zeta potential of −10 mV to −20 mV or 0 to −30 mV.

In some embodiments, the nanoparticles comprise poly (D,L-lactide co-glycolide) (PLGA) and/or polyvinyl alcohol (PVA), and optionally wherein said nanoparticles are composed of about 2-5% of said PVA, and optionally wherein said nanoparticles have a hydrodynamic diameter of 3-30 nm or 3-25 nm (e.g., 3, 4, 5, 6, 10, 15, 25, or 30 nm). In particular embodiments, the compositions comprise 15-60% or 20-50% glucose or other cryoprotectant.

In certain embodiments, provided herein are systems comprising: a) an infusion pump or osmotic pump, which is optionally implanted in a subject; and b) a composition comprising the nanoparticles described herein. In particular embodiments, the nanoparticles are located inside the infusion or osmotic pump.

In some embodiments, provided herein are compositions, systems, or kits comprising: a) a plurality of nanoparticles comprising poly (D,L-lactide co-glycolide) (PLGA), and/or polyvinyl alcohol (PVA), wherein optionally at least part of said PVA is associated with the nanoparticle surface (residual PVA), wherein the nanoparticles encapsulate, or are attached or absorbed to, at least drug agent; and b) a cryoprotectant, which is optionally glucose. In some embodiments, the plurality of nanoparticles and glucose are combined in a mixed-composition. In additional embodiments, the cryoprotectant, and the plurality of nanoparticles are present in the mixed-composition at about a 1:1 w/w ratio. In other embodiments, the cryoprotectant, and the plurality of nanoparticles are present in the mixed-composition at about a 0.4:1-1:0.4 w/w ratio. In certain embodiments, the compositions herein further comprise saline or buffer solution.

In some embodiments, provided herein are lyophilized compositions comprising: a) a plurality of nanoparticles comprising poly (D,L-lactide co-glycolide) (PLGA), and/or polyvinyl alcohol (PVA), wherein the nanoparticles encapsulate, or are attached or absorbed to, at least one drug agent; and b) a cryoprotectant, which is optionally glucose. In further embodiments, the cryoprotectant, and the plurality of nanoparticles are present in the composition at about a 1:1 w/w ratio. In other embodiments, the cryoprotectant, and the plurality of nanoparticles are present in the composition at about a 0.4:1-1:0.4 w/w ratio.

In particular embodiments, provided herein are compositions comprising: a plurality of nanoparticles which optionally comprise poly (D,L-lactide co-glycolide) (PLGA), and/or polyvinyl alcohol (PVA), wherein the plurality of nanoparticles encapsulate, or are attached or absorbed to, at least one drug agent, wherein the nanoparticles optionally have an average TEM diameter of about 10-115 nm or about 60-90 nm or hydrodynamic diameter 3-30 nm or 30 to <350 nm; wherein the at least one drug agent comprises one, or combinations of, the following: i) an antioxidant enzyme, which is selected from: a peroxiredoxin (PRX), a glutathione peroxidase (GPX), ascorbate peroxidase, monodehydroascorbate reductase, dehydroascorbate reductase, and glutathione reductase; ii) an anti-inflammatory drug, which is optionally: ibuprofen, naproxen, diclofenac, celecoxib, mefenamic acid, etoricoxib, indomethacin, aspirin, or a steroid; iii) an angiogenic factor, which is optionally: a VEGF (Vascular Endothelial Growth Factor) family molecule, a FGF (Fibroblast Growth Factor) family molecule, or angiopoietin; iv) a neurotrophic factor, which is optionally: epidermal growth factor, basic fibroblast growth factor, brain-derived neurotrophic factor, and glial cell line-derived neurotrophic factor; v) a prostaglandin or prostaglandin analogue, which is optionally: A) a Prostaglandin F 2α, which is optionally: Xalatan (latanoprost), Zioptan (tafluprost), Travatan Z (travoprost), Lumigan (bimatoprost), or Vyzulta (latanoprostene bunod); B) a Prostaglandin E1, which is optionally Muse (alprostadil), Edex (alprostadil), Caverject (alprostadil), Caverject Impulse (alprostadil), or Cytotec (misoprostol); C) a Prostaglandin E2, which is optionally: Cervidil (dinoprostone), or Prepidil (dinoprostone), and D) a Prostacyclin, which is optionally: Veletri (epoprostenol), Flolan (epoprostenol), Remodulin (treprostinil), Tyvaso (treprostinil), and Ventavis (iloprost); and vi) an Alzheimer's disease drug which is optionally: Aducanumab, Lecanemab, a Cholinesterase inhibitor, Donepezil, Rivastigmine, Galantamine, a Glutamate regulator, Memantine, combination of Donepezil and memantine, Orexin receptor antagonist, and Suvorexant. In particular embodiments, the nanoparticles further encapsulate, or are attached or absorbed to, an additional agent selected from superoxide dismutase (SOD), a catalase, a pore-forming agent, Dimethyl tartaric acid (DMT), a protective agent for encapsulated therapeutic, human serum albumin, PVA associated with nanoparticles to facilitate their dispersion upon reconstitution, and glucose as a cryoprotectant to prevent nanoparticle aggregation and facilitate dispersion in saline or physiological buffer for administration. Different excipients/cryoprotectant like trehalose, sucrose, fructose, glucose, and sorbitol may be used to increase nanoparticles physical stability during freeze-drying to prevent their aggregation, and protect them against the mechanical stress of ice crystals.

In some embodiments, provided herein are methods comprising some or all of the following steps: a) dissolve poly (dl-lactide co-glycolide) (PLGA) in ethyl acetate to generate a PLGA solution, and optionally dissolving dimethyl tartaric acid (DMT) in said PLGA solution; b) dissolve polyvinyl alcohol (PVA) in water to generate a PVA solution, and optionally removing any undissolved PVA by centrifugation and/or filtration; c) at least one of the following: i) dissolve human serum albumin and at least one drug agent (e.g., water soluble drug agent) in water to generate a drug agent solution, and ii) dissolve a drug agent in ethyl acetate to generate a drug agent solution; d) combine said drug agent solution and said PLGA solution to generate a combined solution; e) vortexing or otherwise treating said combined mixture to generate a first water-in-oil emulsion; f) diluting said PVA solution such that a 2-5% PVA w/v solution is generated; g) cooling said 2-5% PVA w/v solution such that is between 1 and 10 degrees Celsius; h) adding ethyl acetate to said 2-5% PVA w/v solution such that it is saturated; i) combining said water-in-oil emulsion with said 2-5% PVA w/v solution to generate a combined solution; f) sonicating said combined solution to generate a sonicated solution; g) passing said sonicated solution through a homogenizer such that a water-in-oil-water (w/o/w) emulsion is generated; h) treating said w/o/w emulsion to evaporate ethyl acetate therefrom to generate a treated w/o/w emulsion comprising nanoparticles; i) freezing and lyophilizing said nanoparticles to remove residual ethyl acetate; j) dispersing said treated nanoparticles in water and to generate a nanoparticles dispersion; k) subjecting said nanoparticles dispersion to tangential flow filtration to remove excess PVA; and 1) lyophilizing nanoparticles following washing steps in TFF to generate a lyophilized nanoparticle powder.

In certain embodiments, the nanoparticles further encapsulate or are attached or adsorbed to, an imaging agent selected from near-infrared dye, fluorescent dye, color dye, radiotracer 18F-fluorodeoxyglucose (FDG) for Single photon emission computed tomography (SPECT) and positron emission tomography (PET), or magnetic resonance (MRI) contrast agents such as gadolinium-based, iron oxide, etc. In certain embodiments, the methods further comprise tracking the imaging agent in the subject (e.g., to determine if the nanoparticles have reached the target tissue in sufficient quantities). In certain embodiments, PET is employed for imaging. In other embodiments, MRI is employed.

In certain embodiments, the at least one drug agent is selected from the group consisting of: a prostaglandin analogue, vascular endothelial growth factor (VEGF), magnesium, an anti-inflammatory drug, acetaminophen, an antioxidant enzyme, a neurotrophic factor, and an angiogenic factor. In further embodiments, the at least one drug agent comprises one, or combinations of, the following: i) an antioxidant enzyme, which is optionally: superoxide dismutase (SOD), a catalase (CAT); a peroxiredoxin (PRX), a glutathione peroxidase (GPX), ascorbate peroxidase, monodehydroascorbate reductase, dehydroascorbate reductase, and glutathione reductase; ii) an anti-inflammatory drug, which is optionally: ibuprofen, naproxen, diclofenac, celecoxib, mefenamic acid, etoricoxib, indomethacin, aspirin, or a steroid; iii) an angiogenic factor, which is optionally: a VEGF family molecule, a Fibroblast (FGF) family molecule, or angiopoietin; iv) a neurotrophic factor, which is optionally: epidermal growth factor, basic fibroblast growth factor, brain-derived neurotrophic factor, and glial cell line-derived neurotrophic factor; v) a prostaglandin or prostaglandin analogue, which is optionally: A) a Prostaglandin F2α, which is optionally: Xalatan (latanoprost), Zioptan (tafluprost), Travatan Z (travoprost), Lumigan (bimatoprost), or Vyzulta (latanoprostene bunod); B) a Prostaglandin E1, which is optionally Muse (alprostadil), Edex (alprostadil), Caverject (alprostadil), Caverject Impulse (alprostadil), or Cytotec (misoprostol); C) a Prostaglandin E2, which is optionally: Cervidil (dinoprostone), or Prepidil (dinoprostone), and D) a Prostacyclin, which is optionally: Veletri (epoprostenol), Flolan (epoprostenol), Remodulin (treprostinil), Tyvaso (treprostinil), and Ventavis (iloprost); and E) an Alzheimer's disease drug which is optionally: Aducanumab, Lecanemab, a Cholinesterase inhibitor, Donepezil, Rivastigmine, Galantamine, a Glutamate regulator, Memantine, combination of Donepezil and Memantine, Orexin receptor antagonist, and Suvorexantm; and F) Inhibitor of BACE1: BACE1 is an aspartic protease that functions in the first step of the pathway leading to the production and deposition of amyloid-D peptide (Aβ). Hence, inhibitors of BACE1 have direct implications in developing AD pathology. Examples of inhibitors of BACE1 are: Verubecestat (MK-8931), Verubecestat (MK-8931), Lanabecestat (AZD3293), Atabecestat (JNJ-54861911), Elenbecestat (E2609), etc.

In further embodiments, the subject has the eye condition and the administration is conducted in or near a lymphatic vessel (e.g., 1-5 mm or 1-10 mm away) on the subject's face, and wherein the eye condition is optionally selected from: a Corneal transplant, Dry eye, Herpes Simplex Virus (HSV-1), keratitis, Glaucoma, an Intraocular tumor, Uveitis, and Retinitis pigmentosa (RP). In some embodiments, the subcutaneous injection is performed at a location on the subject in the vicinity of deep cervical lymph nodes or lymph vessels thereof (FIG. 1), such that the plurality of nanoparticles are taken up by the lymphatic capillaries and are transported to the neurological tissue. In further embodiments, the deep cervical lymph nodes are selected from: Anterior Cervical Lymph Nodes, Deep Cervical Lymph Nodes, and Inferior Deep Cervical Lymph Nodes. In certain embodiments, the subcutaneous injection is performed at a location on the subject within about 15 mm, 12 mm, 10 mm, 8 mm 5 mm or 3 mm or 2 mm of the deep cervical lymph nodes or lymph vessels thereof, such that the plurality of nanoparticles are taken up by lymphatic capillaries and are transported to neurological tissue. In other embodiments, the subcutaneous injection is performed at a location on the subject in the vicinity of the inner canthus lymph vessel, the outer canthus lymph vessel, or the inferior eyelid lymph vessel, such that the plurality of nanoparticles are taken up by lymphatic capillaries and are transported to eye tissue. In additional embodiments, the subcutaneous injection is performed at a location of the subject within about 15 mm, 12 mm, 10 mm, 8 mm 5 mm, or 3 mm of the inner canthus lymph vessel, the outer canthus lymph vessel, or the inferior eyelid lymph vessel, such that the plurality of nanoparticles are taken up by lymphatic capillaries and are transported to eye tissue (FIG. 2). In further embodiments, the subcutaneous or intraperitoneal or intramuscular injection is performed at a location within about 15 mm, 12 mm, 10 mm, 8 mm 5 mm, or 3 mm or 2 mm on the subject in the vicinity of lymph vessels or lymph nodes, such that the plurality of nanoparticles are taken up by lymphatic capillaries and are transported to the neurological tissue or the eye tissue.

In particular embodiments, the administering is via subcutaneous injection, and wherein the subcutaneous site of administration on the subject is warmed and/or massaged before, during, or after the administering. In additional embodiments, the nanoparticles have at least one of the following properties: i) a hydrodynamic diameter range of 30 to <350 or 100 to 300 nm, or about 200-250 nm or nm; ii) a transmission electron microscopic (TEM) diameter of 10-115 nm, or about 60-90 nm; and/or iii) a zeta potential range of −10 mV to −20 mV or 0 to −30 mV.

In certain embodiments, the compositions have a viscosity of about 1.0 to 10 centipoise (cps). In further embodiments, the nanoparticles are present in the composition at a concentration of about 1 to 15 mg/ml, or about 10 mg/ml. In additional embodiments, the composition is administered at an infusion rate of about 20 to 80 ml per hour, and/or wherein the subject is a human, and/or the administering is conducted for about 20-40 minutes.

In particular embodiments, the nanoparticles comprise a material selected from: a polymer, metal, dendrimers, gold, lipids, ceramic, inorganic-based nanomaterial, carbon-based nanomaterial, organic-based nanomaterial, and composite-based nanomaterial. In other embodiments, the subject is a human, and/or the administering provides about 450 mg of the nanoparticles to the subject. In some embodiments, the nanoparticles comprise poly (D,L-lactide co-glycolide) (PLGA) and/or polyvinyl alcohol (PVA).

In particular embodiments, the nanoparticles are formulated using biodegradable polymers, synthetic or natural. For example, biodegradable polymers are polyesters, polylactic acid, poly(lactic-co-glycolic) acid, and poly(caprolactone). PLGA is a co-polymer of polylactic acid and polyglycolic acid; of different molecular weights and compositions (polylactide to polyglycolide ratios), acid terminated or ester terminated. Other biodegradable polymers include Poly(butylene succinate), Poly(p-dioxanone), Polyamides and poly(ester-amide)s, poly(ester urethane)s, Polyanhydrides, etc. Natural biodegradable polymers are called biopolymers such as polysaccharides, as starch and cellulose, Proteins such as collagen, gelatin, Polysaccharides such as Chitin or processed chitin to Chitosan, Polysaccharides such as starch, alginates, Polymers produced by microbes such as Poly(hydroxybutyrate) (PHB), Poly(hydroxybutyrate-co-hydroxyvalerate) (PHBV).

In particular embodiments, the neurodegenerative and/or neurological condition is selected from the group consisting of: Parkinson's Disease, Alzheimer's Disease, Multiple Sclerosis (MS), Huntington's disease, and Amyotrophic Lateral Sclerosis (ALS). In other embodiments, the neurological condition is selected from the group consisting of: traumatic brain injury, ischemia/reperfusion injury, a spinal injury, peripheral nerve injury, and a stroke, brain tumor, epilepsy, neural infection, and meningitis. In other embodiments, the drug agent comprises SOD and catalase and optionally the SOD and the catalase are encapsulated in the same nanoparticles, attached or absorbed to, separate nanoparticles. In certain embodiments, the SOD and the catalase are administered simultaneously to the subject.

In other embodiments, the methods herein further comprise: an additional administering wherein a plurality of the type of nanoparticles are administered intravenously to the subject before, during, or after the initial administering. In certain embodiments, the at least one drug agent comprises a prostaglandin or a prostaglandin analogue and is administered first, and wherein the method further comprises: administering additional nanoparticles that encapsulate, or are attached or absorbed to, one or more antioxidant enzymes which are optionally SOD and/or catalase. In particular embodiments, the nanoparticles are biodegradable.

In certain embodiments, the administering is via continuous infusion over a time period of at least 1 minute, 5 minutes, 30 minutes, 1 hour, two hours or more. In other embodiments, each mg of nanoparticles comprise SOD and is loaded with about 5 μg to about 150 μg SOD; and/or wherein each mg of nanoparticles comprise catalase and is loaded with about 10 μg to about 150 μg catalase. The ratio of SOD to CAT in nano-SOD/CAT formulation can vary from 0.1 to 99.9, or about 1:1 or 1:2 or 1:5 or 1:9 or 2:1, 5:1 or 9:1 or in some embodiment it is SOD only or CAT only.

In some embodiments, the methods further comprise: administering an additional agent that can dissolve or bind amyloid precursor protein (APP), amyloid plaques, an inhibitor of BACE1, and/or an agent to clear the brain lymphatic system to facilitate drainage of metabolic waste from the brain; and optionally wherein such agent is vascular endothelial growth factor, Progestin that dilates lymphatic vessels, or an antibody that binds to APP. In further embodiments, the drug agent comprise SOD and/or, and wherein after the administering, some of the SOD and the catalase is released rapidly from the one or more nanoparticles in the neurological tissue, and then the SOD and the catalase that remains in the one or more nanoparticles is released slowly over a sustained period of time in the neurological tissue. In other embodiments, the methods further comprise: administering one or more cells, growth factors, tissue grafts, antioxidants, hormones, steroids, vitamins, minerals or a combination thereof.

In particular embodiments, the subject has a spinal cord injury, which is optionally a recent spinal cord injury. In some embodiments, the subject is a human. In other embodiments, the administering is performed by a paramedic. In additional embodiments, the neurological tissue comprises the subject's brain. In other embodiments, the subject is a human, and wherein about 2.4 to 9.7 mg/kg of the nanoparticles are administered to the subject.

### DESCRIPTION OF THE FIGURES

FIG. 1 shows the lymph nodes associated with the human neck which includes: Deep Lymph Nodes: 1 Submental and 2 Submandibular (Submaxillary); Anterior Cervical Lymph Nodes (Deep): 3 Prelaryngeal, 4 Thyroid, 5 Pretracheal, and 6 Paratracheal; Deep Cervical Lymph Nodes: 7 Lateral jugular, 8 Anterior jugular, 9 Jugulodigastric; Inferior Deep Cervical Lymph Nodes: 10 Juguloomohyoid and 11 Supraclavicular (scalene).

FIG. 2 shows eye related lymph nodes and vessels including the inner canthus lymph vessel, the outer canthus lymph vessel, and the inferior eyelid lymph vessel.

FIG. 3 shows a hypothetical model of how nanoparticles reach the lymphatic capillaries following their Subcutaneous (SQ) injection and their passage through lymphatic vesicles.

FIG. 4A shows the optimization of tangential flow filtration (TFF) conditions, a relation between Flow Rate and Feed Pressure, for the recovery of nanoparticles.

FIG. 4B shows the number of washing cycles in TFF needed to remove about 95% of free PVA, which is used as an emulsifier.

FIG. 5 shows the effect of different amounts of sugar added in nano-SOD/CAT formulation prior to lyophilization on particle size (A), polydispersity index (B) and time it takes to redisperse formulation (C). It takes only 5 min to redisperse when 1:1 w/w ratio nano-SOD/CAT and glucose were mixed prior to lyophilization (C). Under the microscope, the formulation with 50% glucose (1:1 w/w) looks quite monodispersed, whereas the one with 10% glucose shows the presence of aggregates even at 60 min (D).

FIGS. 6A-C show: (A) Hydrodynamic diameter, (B) Transmission Electronic Microscopic picture, and (C) Zeta potential of nano-SOD/CAT. The mean hydrodynamic diameter is 273.1 nm with a polydispersity index of 0.099, whereas the diameter measured from the transmission electron microscopy picture is =90+6.7 nm (n=20). Zeta potential is −17.21 mV.

FIG. 7 shows the localization of SQ-injected nanoparticles (60 mg/kg) in the brain of the AD mouse model (5XFAD). The AD mouse mode of age 9 to 10 weeks where SQ injected the near-infrared dye-loaded nanoparticles. (A) The optical images of the sections of the brain at 2 and 7 days following SQ injection of nanoparticles. Left images control (no nanoparticles) and right images of the brain sections from animals that received nanoparticles. (B) Quantification of brain signal. Shown is the combined signal from all the brain sections.

FIG. 8 shows the effect of treatment with nano-SOD/CAT (60 mg/kg) on amyloid deposition in the AD mouse model. The treatment was initiated at age 4 weeks when animals do not show amyloid deposition, every week for the first four weeks, and thereafter once in two weeks. The treated animals were evaluated for amyloid β deposition using Maestro at the age of 16 weeks (about 12 weeks post-treatment), and the results were compared with untreated animals. (A) The brain section images show amyloid deposition in different sections of the brain. The red signal represents higher amyloid deposition, whereas blue is the background. (B) The cumulative signal from all the sections of the brain from each group. Groups included untreated, IV-treated and SQ treated. (C) Treated animals show a better novel object recognition index 8 weeks post-treatment than untreated animals. The drop in the novel object recognition index is significant in untreated animals from 4 weeks to 8 weeks, whereas it is not in treated animals.

FIG. 9 shows the effect of treatment with nano-SOD/CAT on Y-Maze. The treatment was given SQ to 5XFAD mice (60 mg/kg) at age of 4-5 weeks, every week for the first four weeks and thereafter once in two weeks. The data shown are comparative % spontaneous alternation at 4 weeks post-treatment or at 8-9 weeks of mice age in different treatment groups (FIG. 9A). There is a reduction in % spontaneous alterations in untreated and IV-treated animals as compared to pre-treatment, whereas the SQ-treated animals retained the same % spontaneous alterations as pre-treatment time, indicating inhibition of progression of cognitive decline in SQ treated group as compared to untreated control (**<0.01). The continuation of the study shows a more rapid decline in % spontaneous alterations in untreated group compared to SQ-treated animals with respect to the pre-treatment values (FIG. 9B). The SQ-treated animals show better recognition of novel objects at 8 weeks as compared to untreated control (FIG. 9C). The decline in recognition index in untreated group from 4 weeks to 8 weeks is greater than in SQ treated animals (FIG. 9C).

FIG. 10 shows the localization of nanoparticles at the lesion site in the rat spinal cord injury model following SQ injection. Near infrared-dye-loaded nanoparticles were injected SQ (30 mg/kg) to the rats. The spinal cords were harvested at 2 days and 7 days post-administration of NPs; the animals were perfused prior to harvesting of spinal cords prior to imaging (FIG. 10A) using Maestro optical imaging system and for the signal count (FIG. 10B). Following imaging of the spinal cord, the lesion sites were homogenized; the homogenates were loaded into 96-well plated and imaged for the total signal count (FIG. 10C). The data show increasing localization of nanoparticles at the lesion site with time post-administration of nanoparticles.

FIG. 11 shows the relative signal due to near-infrared dye-loaded nanoparticles in the retinal tissue following SQ, IP, and IV administration of nanoparticles (120 mg/kg) in rd10 mice of RP. The retinal tissues were harvested and homogenized for signal measurements, and the data are normalized to per mg tissue weight. SQ injection resulted in a higher signal due to nanoparticles as compared to that with IP and IV injection of nanoparticles.

FIG. 12 shows: (A) expression of CAT and SOD in retinal tissue in rd10 mouse model of RP. Retinas were harvested at P21 following treatment with nano-SOD/CAT dose at 120 mg/kg given at pup at age P6, P12, and P18 intraperitoneally. Data shown are mean±standard error mean. (B) Analysis of retina from the above-treated animals for outer nuclear layer (ONL) thickness using optical coherence tomography (OCT) in rd10 mouse model of RP, n=12; p=0.001; (C) Scanning laser ophthalmoscopy (SLO) images of mouse retinal and quantification of white spots. Data shown are mean±standard error mean, n=5-7.

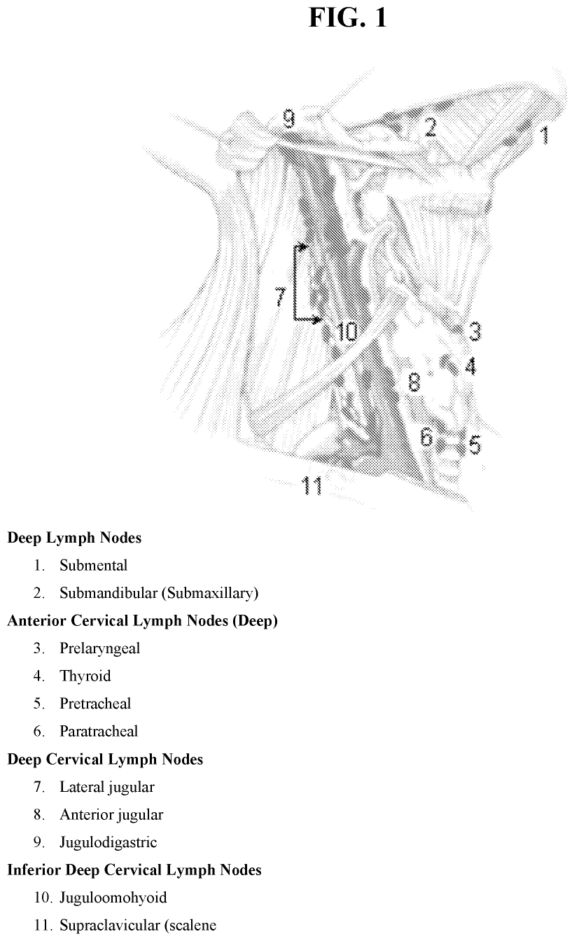

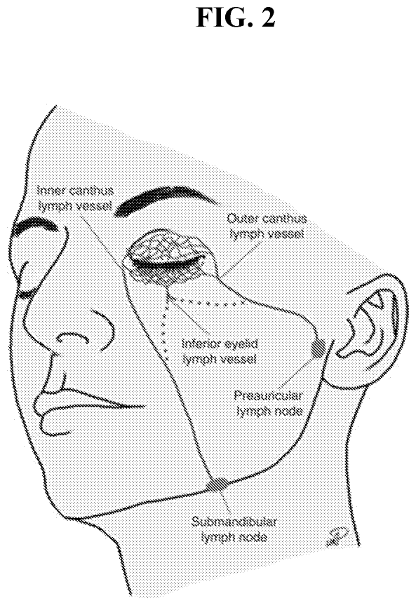

### DESCRIPTION OF THE TABLES

Table 1 shows the formulation composition, conditions for TFF, and physical characterization of nano-SOD/CAT formulated using bovine source antioxidant enzymes.

Table 2 shows the formulation composition, conditions for TFF, and physical characterization of nano-SOD/CAT formulated using recombinant human antioxidant enzymes. The formulation of nano-SOD/CAT is prepared with two different doses of antioxidant enzymes encapsulated.

Table 3 describes the effect of reverse flow on improving the yield of nano-SOD/CAT.

Table 4 shows the enzymatic activity of nano-SOD/CAT for SOD and CAT.

Table 5 shows that nano-SOD/CAT meets certain key requirements for parenteral products as per the USP guidelines.

### DETAILED DESCRIPTION

Provided herein are composition, systems, kits, and methods for preventing and/or treating, reversing, and/or inhibiting progression of, a neurodegenerative and/or neurological conditions and/or eye conditions (e.g., retinitis pigmentosa) by administering a plurality of nanoparticles to a subject via subcutaneous, intramuscular, or intraperitoneal injection such that the plurality of nanoparticles are taken up by lymphatic capillaries and are transported to neurological and/or eye tissue of the subject via the subject's lymphatic system, wherein the plurality of nanoparticles encapsulate, or are attached or absorbed to, at least one drug agent.

In certain embodiments, provided herein are methods for efficient delivery of nanoparticles encapsulating antioxidant enzymes, such a nano-SOD/CAT to the brain to mitigate oxidative stress, thus regaining the redox balance, ultimately protecting the brain from developing and even reversing the pathology associated with neurological conditions, such as AD. To achieve efficient delivery of nano-SOD/CAT to the brain, one can employ the lymphatic system in the skin and the brain as a pathway to delivering therapeutics to the brain. This approach, which we designate as the “backdoor entry,” bypasses the blood-brain barrier that limits the transport of therapeutics to the brain with conventional oral/intravenous routes of administration.

Oxidative stress is implicated in the onset and progression of Alzheimer's Disease (AD) by promoting multiple post-translational modifications of proteins, oxidative DNA damage, and lipid peroxidation. The oxidative damage accelerates the expression of beta-secretase-1 (BACE1), an enzyme involved in Amyloid-β (Aβ) generation. In turn, increased Aβ leads to increased oxidative stress and mitochondrial dysfunction. This vicious feed-forward cycle is repeated, causing progressive neurodegeneration in AD. In fact, mitochondrial dysfunction is considered one of the most prominent features observed in vulnerable neurons of the AD patients' brain samples.

Compelling evidence presenting disrupted oxidant/antioxidant balance in AD led to the formulation of a hypothesis that compounds scavenging free radicals that cause oxidative stress and/or boosting oxidative stress defense mechanisms might provide therapeutic benefits in treating AD. Therefore, natural and synthetic antioxidants, including antioxidants targeting mitochondria, have been tested. Despite some promising results in vitro and animal models of AD, clinical translation of antioxidants as a therapy to treat or halt the disease progression remains generally elusive. The issues are the low bioavailability of antioxidants, their instability, limited transport to the target tissue, the presence of blood-brain barrier (BBB) that limit the transport of therapeutics to the brain, and/or poor antioxidant capacity due to their “noncatalytic” mechanism of action. As a result, antioxidants such as vitamins, flavonoids, antioxidant mimetics, etc., which are noncatalytic, become inactive once they interact with free radicals; hence their therapeutic levels drop rapidly. Repeated and high dosing of antioxidants to maintain their therapeutic levels in the brain is thus challenging in humans because of toxicity concerns _{[1]}. In line with that, the administration of high Vitamin E doses has been reported to increase the risk of mortality.

Provided herein are methods of delivery of nano-SOD/CAT to the brain to mitigate oxidative stress, thus regaining the redox balance, ultimately protecting the brain from developing and even reversing the pathology associated with AD. Antioxidant enzymes such as SOD and CAT are catalytic in the mechanism of action, hence are highly effective in neutralizing free radicals/reactive oxygen species (ROS) and regaining the redox balance. However, due to their short half-lives (5-11 min) due to their rapid clearance, exogenously delivered antioxidant enzymes are ineffective in combating oxidative stress. Hence, encapsulation of antioxidant enzymes protects and sustains their effect. To achieve efficient delivery of nano-SOD/CAT to the brain, the lymphatic system in the skin and the brain is employed as a pathway to delivering therapeutics to the brain. In addition to SOD and CAT, other antioxidant enzymes include peroxiredoxins (PRXs), glutathione peroxidases (GPXs), and the four enzymes of the ascorbate-glutathione pathway (e.g., ascorbate peroxidase, monodehydroascorbate reductase, dehydroascorbate reductase, and glutathione reductase), play a vital role in detoxifying ROS.

In certain embodiments, the nanoparticles herein encapsulate or are attached or absorbed to, a prostaglandin or prostaglandin analogue (e.g., Latanoprost). Latanoprost is a prostaglandin F-2 alpha (FP) analog that is used to reduce intraocular pressure in patients with open-angle glaucoma. Latanoprost reduces intraocular pressure by increasing the outflow of aqueous humor through the uveoscleral pathway, which is the principal site of action of prostanoids _{[2]}. Latanoprost is a prodrug that is converted within the cornea to an active metabolite by esterases and is applied as ophthalmic drops. There is evidence that latanoprost increases lymphatic drainage from the eye. Prostaglandin analogs are well-established to stimulate lymphatic drainage in non-ocular tissues _{[3]}. Cellular changes induced by latanoprost may be implicated in this process. Impaired lymphatic drainage in the brain is implicated in aging and Alzheimer's Disease _{[4]}. Lymph is the colorless fluid in specialized vessels that carries immune cells and waste like toxic compounds and cellular debris. The buildup of a protein called amyloid-beta in the brain is a hallmark of Alzheimer's Disease _{[5]}.

In particular embodiments, nanoparticles with an antioxidant enzyme (e.g., SOD and/or Catalase) and a prostaglandin or prostaglandin analogue should produce complementary effects by facilitating lymphatic drainage and as well as contract the oxidative stress condition in neurodegenerative diseases. In addition, the compositions could help in treating ocular conditions in managing intraocular pressure as well as protecting retinal tissue from oxidative stress conditions. In some embodiments, a therapeutic strategy includes the sequential administration of nanoparticles, first nanoparticles containing prostaglandin-type molecules to clear the lymphatic system, followed by the administration of nanoparticles containing therapeutic agents such as antioxidant enzymes, anti-inflammatory drugs, neurotrophic growth factors to promote neurogenesis, angiogenic drugs to promote vascularization. The nanoparticles could be formulated containing one agent or with a combination, or they could be prepared separately and combined prior to administration.

The eye conditions include Blepharitis, Blepharoptosis, Chorioditis, Conjunctivitis, Corneal Abrasion, Corneal Cystine Crystal Accumulation, Eye Redness, Eye Redness/Itching, Eyelash Hypotrichosis, Glaucoma, Herpetic Keratitis, Hordeolum, Inhibition of Intraoperative Miosis, Iritis, Macular Degeneration, Macular Edema, Myopic Choroidal Neovascularization, Neuromyelitis Optica, Ocular Fungal Infection, Ocular Rosacea, Pupillary Dilation, Refraction,

### Retinal Disorders, Retinopathy, Strabismus, Vitreomacular.

Eye care medications include, but not limited to Acetazolamide for glaucoma (Diamox, Eytazox), Acetylcysteine for dry eyes (Ilube), Aciclovir, Antazoline and xylometazoline eye drops (Otrivine-Antistin), Apraclonidine eye drops (Iopidine), Atropine eye drops (Minims Atropine), Azelastine eye drops for allergies (Optilast). Anti-VEGF therapies, initially introduced for the treatment of choroidal neovascularization in patients with age-related macular degeneration, have also been shown to have impact on the management of retinal vascular disease and are currently an indispensable component for the treatment of macular edema in patients with diabetic eye disease and retinal vein occlusions.

Anti-inflammatory drugs can be used as the drug agent herein, and include, for example, ibuprofen, naproxen, diclofenac, celecoxib, mefenamic acid, etoricoxib, indomethacin, aspirin, steroids, etc. The most potent angiogenic factors to promote vasculogenesis and angiogenesis in the placenta include VEGF family molecules, FGF family molecules, angiopoietin, which may be used as the drug agent herein. Neurotrophic factors are endogenous substances that control cell proliferation and differentiation in the nervous system and can be used as the drug agent herein. These factors include, for example, the epidermal growth factor, basic fibroblast growth factor, brain-derived neurotrophic factor, and glial cell line-derived neurotrophic factor.

In certain embodiments, antioxidant enzymes can be used as the drug agent herein and include, for example, the superoxide dismutases (SODs) (e.g., human SOD) catalases (e.g., human catalase), peroxiredoxins (PRXs), glutathione peroxidases (GPXs), and the four enzymes of the ascorbate-glutathione pathway, which include: ascorbate peroxidase, monodehydroascorbate reductase, dehydroascorbate reductase, and glutathione reductase.

In some embodiments, prostaglandin analogs are used as the drug agent herein. Prostaglandin analogues are a class of drugs that mimic the function of naturally occurring prostaglandins, which are hormones derived from phospholipids in the body. When prostaglandins bind to prostaglandin receptors on the cell surface, they affect different biological processes. For example, prostaglandin biosynthesis plays a role in pain and inflammation, controlling eye pressure, stomach acid production, and inducing labor during pregnancy. Because prostaglandins have many different functions in the body, synthetic prostaglandins are also used for a wide variety of diseases. The most common treatment is for glaucoma, but prostaglandin analogs can also treat stomach ulcers, erectile dysfunction, pulmonary hypertension, and induce labor. There are four types of prostaglandins. Examples of prostaglandin analogs of different types are as follows. Type Prostaglandin F 2α: These drugs primarily have an effect on the eye. For example, Xalatan (latanoprost), Zioptan (tafluprost), Travatan Z (travoprost), Lumigan (bimatoprost), and Vyzulta (latanoprostene bunod). Type Prostaglandin E1: Prostaglandin E1 (PGE 1) is also known as alprostadil. It is most effective for treating erectile dysfunction by opening blood vessels to increase vascular blood flow. For example, Muse (alprostadil), Edex (alprostadil), Caverject (alprostadil), Caverject Impulse (alprostadil), and Cytotec (misoprostol). Type Prostaglandin E2: Prostaglandin E2 (PGE 2), also known as dinoprostone, increases uterine contractions, opens blood vessels, and prepares the cervix for labor and delivery during pregnancy. For example, Cervidil (dinoprostone), and Prepidil (dinoprostone). Type Prostacyclins: Prostacyclins (also called prostaglandin I2 or PGI 2) are specific types of prostaglandins that have an important role in respiratory diseases. In healthy people, Prostacyclins relax blood vessel walls and allow blood to flow freely in the lungs. People with pulmonary arterial hypertension don't make enough natural prostacyclin, causing narrowing of blood vessels and high blood pressure in the lungs. Prostacyclin drugs are an important treatment for this disease. For example, Veletri (epoprostenol), Flolan (epoprostenol), Remodulin (treprostinil), Tyvaso (treprostinil), and Ventavis (iloprost).

In some embodiments, the nanoparticles with the drug agent via the lymphatic system are used to treat eye conditions. The eye was thought to lack lymphatic vessels except for the conjunctiva. However, recent studies have shown that the corneal limbus, ciliary body, lacrimal gland, orbital meninges, and extraocular muscles contain lymphatic vessels (and are targets for subcutaneous administration herein) and that the choroid might have a lymphatic-like system. Lymphatics and lymphangiogenesis in the eye are attributed to various ocular diseases. Immunological staining with lymphatic-specific markers as well as histological examinations, has revealed the distribution of lymphatic vessels in the eye. Conjunctiva is well known to possess lymphatics. The cornea limbus, ciliary body, lacrimal gland, orbital meninges, and extraocular muscle also contain lymphatic vessels, and the choroid might have a lymphatic-like system.

The compositions, systems, and methods herein, in certain embodiments, are used for treating eye conditions, particularly lymphatic-associated ocular conditions (e.g., diseases). In certain embodiments, the eye conditions are selected from the following: i) corneal transplant: Lymphatic vessels but not angiogenic vessels are important for the immune rejection; ii) dry eye: it is a low-grade corneal inflammatory disorder induces lymphangiogenesis; iii) HSV-1 keratitis: Corneal herpes simplex virus-1 infection induces lymphangiogenesis via VEGF-A; iv) Glaucoma: “Uveolymphatic pathway”; lymphatics exist in the ciliary body; v) Intraocular tumors “Tumor-associated lymphangiogenesis” correlates the malignancy; and vi) Retinitis pigmentosa.

Clearance of ocular fluid and metabolic waste is a critical function of the eye in health and disease. The eye has distinct fluid outflow pathways in both the anterior and posterior segments. Although the anterior outflow pathway is well characterized, little is known about posterior outflow routes. Recent studies suggest that lymphatic and glymphatic systems play an important role in the clearance of fluid and waste products from the posterior segment of the eye. The glymphatic (glial-lymphatic) system is a glial cell-dependent perivascular network that subserves a lymphatic-like function by clearing metabolic waste from the brain parenchyma _{[6]}. Growing evidence suggests that ocular lymphatic and glymphatic systems are involved in the pathogenesis of a number of disorders associated with ocular fluid homeostasis and waste clearance. Current knowledge of ocular lymphatic and glymphatic systems and their role in retinal degenerative diseases _{[7]}, including Glaucoma, Age-Related Macular Degeneration, Ocular inflammatory diseases such as uveitis, are being investigated _{[8]}.

Retinitis pigmentosa (RP) is a major cause of visual impairment and blindness, affecting millions of people worldwide _{[9]}. RP is the leading cause of inherited blindness, with a prevalence of approximately 1 in 4,000 individuals. According to The Foundation Fighting Blindness, there are about 100,000 people with RP in the United States, and RP is categorized by the National Institutes of Health as a rare disease. RP is caused by a vast array of different gene mutations and has highly variable disease presentations and severities. RP is typically diagnosed in young adulthood when vision loss becomes apparent, but the age of onset may occur much earlier. Despite advances in diagnosis and genetics, effective treatments for RP are not available.

Over the last 20 years, a number of experimental therapies have been evaluated in animal models, including (a) transplantation of retina/retinal pigment epithelial/stem cell, (b) pharmacological treatment with high dose vitamin A, lutein, docosahexaenoic acid, or calcium channel blockers, and (c) gene therapy designed to restore wild type gene function or to upregulate the expression of protective molecules. The translation of these efforts to widespread clinical application is challenging, particularly in view of the large number of different genetic subtypes that comprise RP and because the known genes do not account for all cases of RP.

Increasing evidence suggests that oxidative stress contributes to the pathogenesis of RP _{[10]}. Despite the diversity of retinal degeneration disorders, apoptosis of photoreceptors seems to be a feature common to all. The retina is particularly sensitive to oxidative damage because of its high oxygen demand, its high content of unsaturated lipids, and its exposure to light. Light and oxygen are essential for vision, but paradoxically these elements also trigger the formation of reactive oxygen species (ROS) that can damage retinal tissues. In response to these challenges, the retina has evolved highly efficient defense mechanisms to protect against photo-induced damage. This redox balance is, however, disrupted in retinal diseases, including RP, and this imbalance contributes to photoreceptor cell death _{[11]}. Many RP genes are expressed selectively or predominantly in rod photoreceptors, which mediate vision in low-light conditions. RP thus first manifests as reduced or absent night vision, as rods become dysfunctional and die. Rod cell death leads to cone death and progressive loss of daylight vision, visual acuity, and a narrowed visual field. Although not all of the mechanisms that lead to cone death are known, one important contributor is oxidative stress. Cones have been shown to have oxidized lipids, nucleic acids, and proteins in RP. RP patients present a reduced ocular antioxidant status and an imbalance of the antioxidant-oxidant status in the peripheral blood.

Free radicals are formed as a part of normal metabolic activities but are neutralized by the endogenous antioxidants present in cells/tissue, thus maintaining the redox balance. This redox balance is disrupted in certain neuropathophysiological conditions, causing oxidative stress. Mitochondrial dysfunction under oxidative stress is considered one of the most prominent features in neurodegenerative diseases such as Alzheimer's disease (AD), Parkinson's disease (PK), Huntington's disease (HD), Amyotrophic Lateral Sclerosis (ALS), Ischemia/reperfusion injury, spinal cord injury, ocular, and others _{[12]}. Although the etiology of different neurodegenerative diseases may vary, they share common factors of increased nitroxidative stress and mitochondrial dysfunction that leads to a self-propagating inflammatory cascade of progressive degenerative events _{[13]}.

AD is a devastating, progressive neurodegenerative disorder presenting cognitive decline and memory impairments _{[14]}. It is the most common cause of dementia, a tremendous socioeconomic burden, and remains an incurable disorder. In a vicious cycle, oxidative stress increases Aβ production/secretion that reciprocally promotes oxidative stress, inflammation, and dysfunctional mitochondria _{[15]}. Therefore, targeting oxidative stress is considered central to breaking the self-replicating vicious cycle that leads to progressive neurodegeneration. Furthermore, as oxidative stress is a common pathophysiological process in multiple neurodegenerative diseases, an effective treatment based on an antioxidant delivery system would have broad therapeutic applicability in many clinical settings.

Since the discovery of the lymphatic system in the brain in 2015, named the meningeal lymphatic system (MLS) that surrounds the brain and works in conjunction with the BBB and the cerebrospinal fluid (CSF), its role has been attributed primarily to removing metabolic waste from the brain _{[16]}. Impaired functioning of the MLS is considered to accumulate toxic amyloid Aβ protein in the brain parenchyma, thus aggravating the AD-related pathology. It has also been discovered that the immune cells present in the peripheral immune system are found in the brain parenchyma, suggesting that MLS has a two-way passage _{[17]}. One from the brain parenchyma to the lymphatic system to efflux out metabolic waste, and the second from the peripheral system to the brain parenchyma for immune cells migration to modulate immune response. The discovery of MLS also disrupted the long-held dogma that the brain is an immune privilege site.

In some embodiments, nanoparticles delivered successfully into the lymphatic system in the skin will find their way to the brain parenchyma via MLS. Skin is rich in lymphatic vessels with sufficient gaps in their endothelial lining. SQ injected nanoparticles can pass through these gaps and then can be transported along with the lymphatic fluid to the brain. This approach could bypass the BBB that impedes the transport of intravenously injected therapeutics and nanoparticles. As such, in certain embodiments, FDA-approved biodegradable/biocompatible polymer and human recombinant antioxidant enzymes, or other drug agents, are employed. Further, SQ administration of the treatment at a periodic time interval (e.g., once a month) for treating chronic neurodegenerative diseases such as AD, or eye diseases, may be performed.

In certain embodiments, a neurodegenerative condition, such as a neurodegenerative disease, is treated with the compositions, systems, and methods herein. A neurodegenerative disease is caused by the progressive loss of structure or function of neurons, in the process known as neurodegeneration. Such neuronal damage may ultimately involve cell death. Neurodegenerative diseases include amyotrophic lateral sclerosis, multiple sclerosis, Parkinson's disease, Alzheimer's disease, Huntington's disease, multiple system atrophy, and prion diseases. Neurodegeneration can be found in the brain at many different levels of neuronal circuitry, ranging from molecular to systemic. Because there is no known way to reverse the progressive degeneration of neurons, these diseases are considered to be incurable; however, research has shown that the two major contributing factors to neurodegeneration are oxidative stress and inflammation. Biomedical research has revealed many similarities between these diseases at the subcellular level, including atypical protein assemblies (like proteinopathy) and induced cell death. It is estimated that 50 million people worldwide suffer from neurodegenerative diseases and that by the year 2050, this figure will increase to 115 million people.

Apart from treatment for neurodegenerative diseases as mentioned above, effective delivery of drug agent-nanoparticles, such as antioxidant nanoparticles, could be used in traumatic brain injury (TBI), stroke, spinal cord injury, peripheral nerve injury, ocular conditions (e.g., neuro-visual disorders, photoreceptor degeneration), because, for example, injury progression is due to oxidative stress. In addition to oxidative stress-related neurological conditions, the drug-agent nanoparticles herein could be used for the delivery of other drug agents in treating other neurological conditions. Examples include for example, brain tumor (e.g., anticancer drugs), epilepsy (e.g., eslicarbazepine acetate, Brivaracetam, Cannabidiol, Diazepam, etc.), and infections such as meningitis (e.g., antibiotics, steroids). In addition to the delivery of antioxidants, one could deliver other agents that can dissolve amyloid precursor protein (APP), amyloid plaques, and/or agents to clear the brain lymphatic system (e.g., vascular endothelial growth factor) to facilitate drainage of metabolic waste from the brain.

The present disclosure provides for delivery of the nanoparticles herein via the lymphatic system (e.g., via subcutaneous injection) in order to treat neurodegenerative and neurological conditions, as well as eye conditions. The lymphatic system is composed of a network of vessels throughout the body, superficial and deep, but initial lymphatic vessels are just underneath the epidermal layer of the skin and make up 80% of the total lymphatic vessels in our body _{[18]}. Further, lymph flow from the skin is ˜11-fold higher than that from muscles _{[19]}. In addition, the peritoneal cavity contains a lymphatic capillaries system.

Due to the transport and diffusional barriers between the vascular compartment (blood) and the lymphatic system, intravenously administered nanoparticles do not accumulate in the lymphatic system. To maintain fluid balance, the interstitial fluid produced by vascular bed capillaries in the tissue is taken up by the lymphatic capillaries. However, only small molecules, salts, and metabolites can diffuse from the vascular bed to the interstitial space. Only particles that are significantly smaller <10 nm can escape from the vascular compartment (with significantly low efficiency) into the interstitial fluid, yet nanoparticles (NPs) typically used for drug delivery are far larger >10 nm. Therefore, intravenously administered nanoparticles cannot get into the lymphatic system. Further, opsonization of intravenously administered NPs triggered by complement activation leads to their uptake by mononuclear phagocyte system (MPS) and rapid clearance by the organs of the reticuloendothelial system (RES) (e.g., liver, lung, spleen). Efforts to overcome these barriers either have an insignificant effect or introduced other contending factors. The classic example in this regard is PEGylation of NPs; intended to reduce opsonization and recognition by MPS. Although PEGylation increases the half-life of NPs or PEGylated antioxidant enzymes in circulation, but PEG hampers their extravasation from the vascular bed to the interstitial space. In addition, a biological barrier such as the BBB interferes in the transport of such NPs from the circulation to the brain. The PEG associated with PEGylated NPs interferes in extravasation from circulation to the tissue because of the steric hindrance.

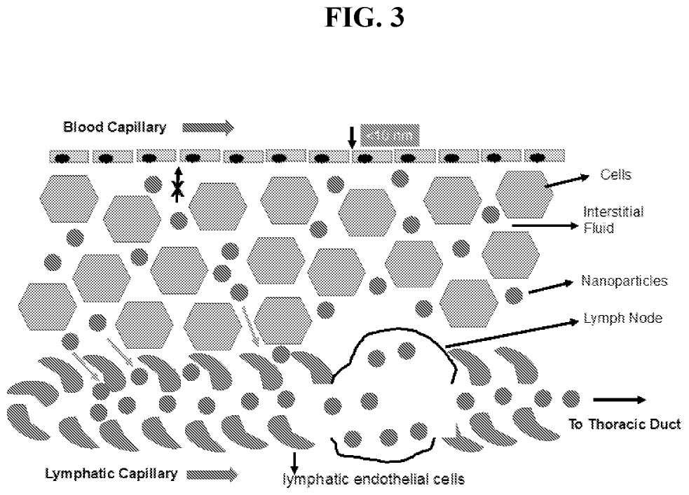

Lymphatic capillaries are composed of a single layer of thin-walled, nonfenestrated lymphatic endothelial cells (LECs) that have poorly developed basement membranes and lack tight and adherens junctions. Lymphatic capillaries are extremely porous because of the gaps between LECs. Also, several hydrophilic channels are present in the tissue space. Hence, NPs ˜100 nm can pass through these channels in the interstitial fluid and via openings in LECs into the lymphatic system (FIG. 3). Lymphatic fluid moves through lymphatic capillaries slowly (˜5 L/day in humans), guided by valves that open only in one direction and then it enters the systemic circulation via the thoracic duct (left side) and right lymphatic duct into the left and right subclavian veins, respectively _{[20]}. The brain lymphatic systems function physiologically as a route of drainage for interstitial fluid (ISF) from brain parenchyma to nearby lymph nodes. Brain lymphatic drainage helps maintain water and ion balance of the ISF, waste clearance, and reabsorption of macromolecular solutes.

As a part of evolution, the lymphatic system, similar to the vascular system and other organs, is also developed differently in various animal species (e.g., mice, rats, rabbits) than in humans. In general, lower mammals have fewer lymph nodes (e.g., ˜22 in mice vs. 500-600 in humans) but mice and humans do have a similar lymphatic trunk—a collection of lymph vessels that carries lymph—a mechanism of lymph flow collection, its flow through the lymph capillaries, and pouring of lymphatic fluid into the thoracic duct _{[21]}. In fact, SQ administered anticancer antibodies which pass through the lymphatic system are tested in different animal species, including in mice for bioavailability, rate of absorption, and biodistribution, and that data are used for designing human clinical trials _{[22]}. Hence, the data obtained in mouse models via lymphatic delivery of NPs could be treated in the same way, and also to determine human equivalent dose using the co-relation (Km) factor that is commonly used for IV and orally administered drugs _{[23]}. Further, large volumes of fluid can be infused slowly into the dermal layer with an infusion pump, a method already being used for delivery of anticancer antibodies _{[22]} and nutrients _{[22]}; hence, SQ administration of NPs in humans (˜5-10 ml) will not pose an issue.

The present disclosure is not limited by the nature of the nanoparticles (NPs). NPs that are sized and will travel in lymphatic vessels are contemplated. One can determine how various nanoparticles with different physical properties interact with lymphatic fluid. For example, and experiment could be as simple as mixing a dispersion of NPs with lymphatic fluid and measuring aggregate formation, and could be the first determinant for NP selection for further evaluation in vivo, as NPs which aggregate in the presence of lymphatic fluid are less likely to navigate through the interstitial fluid to lymphatic vessels. NPs with different zeta potentials can be formulated by modulating the surface. For example, for PVA (anionic) and cationic surfactant (DMAB, Didodecyldimethylammonium ammonium bromide) can be employed along with PVA (neutral and cationic NPs) during emulsification. There is significant flexibility in further modulating size and/or zeta potential of NPs by selecting appropriate energy input and/or selection of the appropriate amount of DMAB along with PVA.

In certain embodiments, the nanoparticles are formulated with antioxidant enzymes. For example, the following may be employed for generating SOD and catalase nanoparticles. Recombinant human SOD and CAT (Obtained from BioVision or Creative BioMart) are significantly more potent in their catalytic activities than the respective bovine-sourced enzymes obtained from Sigma. Human CAT is 5.7-fold more potent than bovine CAT whereas human SOD is 8.1-fold more potent than bovine source SOD when data are normalized to per mg. In certain embodiments, SOD is an enzyme that catalyzes the conversion of superoxide to H₂O₂ and catalase is an enzyme that further degrades H₂O₂ to molecular oxygen and water. The SOD and catalase for use in the methods and compositions of the invention can be obtained from a variety of sources. In a particular embodiment, the SOD and/or catalase are mammalian SOD and/or mammalian catalase, and/or recombinant catalase. As used herein “mammal” and/or “mammalian” refer to a primate, canine, feline, rodent, and the like. Specific examples of mammalian SOD and/or catalase include human, pig, dog, cat, horse, cow, sheep, goat, rabbit, guinea pig, rats and mice SOD and/or catalase.

In particular embodiments, isolated SOD and/or catalase is used in the methods and compositions of the present invention. As used herein, “isolated,” “purified,” “substantially pure or purified” or “substantially isolated”, human recombinant refers to SOD and/or catalase (e.g., mammalian SOD and/or catalase) that is separated from the complex cellular milieu in which it naturally occurs, or chemical precursors or other chemicals when chemically synthesized or prepared using recombinant technology. In some instances, the isolated or purified SOD and/or catalase comprises, consists essentially of, or consists of SOD and/or catalase or combinations thereof. Preferably, isolated or purified SOD and/or catalase comprises at least about 50%, 55%, 60%, 65%, 70%, 75%, 80%, 85%, 90%, 95%, 98% or 99% (on a molar basis) of all macromolecular species present. Modified enzymes such as PEGylated or conjugated to peptides such as TAT peptide could also be encapsulated in, or attached or absorbed to, nanoparticles.

In the methods and compositions of the present invention, the SOD and catalase are encapsulated in, attached to or absorbed to, one or more nanoparticles (NPs). A NP is a microscopic particle whose size is measured in nanometers (nm). Nanoparticles are below micron size, typically in the size range of 100 to <350 nm in diameter so that they can easily pass through openings in the lymphatic capillaries. As will be appreciated by those of skilled in the art, a nanoparticle can be a particle of about 1 nm, a particle of about 10 nm, a particle of less than about 100 nm, or particles smaller than 1000 nm. The size of the nanoparticle will depend upon a variety of factors, which include, for example, the indication for which it is being used and the individual to whom it is being administered. The combination of nanoparticles of different sizes can also be mixed and used. The small-sized drain into the lymphatic system from the site of administration (skin, muscle, or intraperitoneal cavity) more rapidly than large-sized nanoparticles.

In particular aspects, the nanoparticle is biodegradable. In other aspects, the NPs are porous so that the ROS can diffuse into nanoparticles and becomes neutralized. Or encapsulated enzymes are released to neutralize ROS in the tissue. The effect could be due to the combination of the above two mechanisms, i.e., diffusion of ROS into NPs and release of enzymes from NPs to neutralize ROS.

The NPs for use in the methods and compositions provided herein can be made from a variety of compounds. In a particular aspect, the NP is a polymer-based matrix (solid structure) or nanogel. Examples of suitable polymers include poly (D,L-lactide co-glycolide) (PLGA), polylactide (PLL), modifications of these polymers (e.g., polyethylene glycol), or a combination thereof. Polymer nanogels (NGs) are aqueous dispersions of nanosized hydrogel particles, which usually formed through physical or chemical cross-linking of polymer chains which simultaneously demonstrate the features of hydrogels and nanoparticles. The NGs are three-dimensional nanonetwork structures and can be fabricated from a variety of synthetic or natural polymers and a blend thereof. Nanoparticles could be dendrimers, micelles, polymer complexes, etc. Examples of polymers used for nanogel synthesis include N-isopropylacrylamide (NIPAM), poly(N-isopropylacrylamide) (PNIPAM), etc. The NPs can comprise further components. For example, the polymer-based nanogel comprises polyvinyl alcohol and/or L-tartaric acid dimethyl ester. Different types of nanoparticles (e.g., dendrimers, lipid-based) can be formulated for lymphatic delivery.

In certain embodiments, the amount of SOD and catalase, or other drug agent, that can be loaded into the NPs will vary depending upon a variety of factors such as the condition/disease for which the SOD and catalase are being administered, the condition of the individual (e.g., health, age, weight, severity of the condition/disease, etc.), acute or chronic, and the like. In one aspect, each of the one or more nanoparticles comprising SOD, or other drug agent, is loaded with about 5 or 10 μg to about 150 μg SOD or other drug agent. In other aspects, each of the one or more NPs are loaded with about 10, 20, 30, 40, 50, 60, 70, 80, 90, 100, 110, 120, 130, 140, 150, 160, 170, 180, 190, or 200 μg SOD or other drug agent. In another aspect, each of the one or more nanoparticles comprising catalase is loaded with about 10 g to about 150 g catalase. In other aspects, each of the one or more NPs are loaded with about 10, 20, 30, 40, 50, 60, 70, 80, 90, 100, 110, 120, 130, 140, 150, 160, 170, 180, 190, or 200 μg catalase.

In particular aspects, the NPs comprising SOD and catalase, or other drug agent, are formulated such that after administration, some of the SOD and the catalase, or other drug agent, is released rapidly from the one or more nanoparticles, and then the SOD and the catalase, or other drug agent, that remains in the one or more nanoparticles is released slowly over a sustained period of time. For example, enzymes at the nanoparticle interface are released first at a faster rate, which is typically referred to as a burst phase or release. After this initial release, the remaining enzymes are released slowly as the nanoparticle breaks down. Burst release can take place over 1-3 days and release of the remaining enzymes occurs over several weeks. In a particular aspect, the release of the remaining enzymes occurs over 4-6 weeks. The SOD and the catalase, or other drug agent, can be encapsulated in (or attached to or absorbed to) separate nanoparticles. Alternatively, each of the nanoparticles can comprise SOD and catalase (each nanoparticle can comprise both SOD and catalase).

The SOD and the catalase (or two other drug agents) can be administered simultaneously or sequentially. In a particular aspect, the SOD and the catalase (or two other drug agents) are administered simultaneously (e.g., as a single dose) to an individual. In another aspect, first, the catalase is administered to the individual, then the SOD (or other drug agent) is administered to the individual. In yet another aspect, first, the SOD (or other drug agent) is administered to the individual, then the catalase (or second drug agent) is administered to the individual.

In the methods of the invention in which the SOD and catalase (or two other drug agents) are administered sequentially, the period of time between administration of the catalase and SOD (or other drug agents) will vary and can occur immediately, over several minutes, hours, days, weeks, months, years, etc. after a spinal cord injury, the occurrence of a neurodegenerative disease or a neuronal injury. In addition, one or more excipients (vehicles) can be administered in between the sequential dose(s) of SOD and catalase (or two other drug agents). In a particular aspect, one or more excipients are administered after the catalase (or other drug agent) is administered and before the SOD (or second drug agent) is administered to the individual. In another aspect, one or more excipients are administered after the SOD (or other drug agent) is administered and before the catalase (or other drug agent) is administered to the individual. A variety of excipients can be used. Examples of excipients include water, saline, cornstarch, lactose, talc, glucose, magnesium stearate, sucrose, gelatin, and calcium stearate.

The nanoparticles for use in the methods described herein can be formulated with a physiologically acceptable carrier or excipient to prepare a pharmaceutical composition. The carrier and composition can be sterile. The formulation should suit the mode of administration. Suitable pharmaceutically acceptable carriers include but are not limited to water, salt solutions (e.g., NaCl), saline, buffered saline, alcohols, glycerol, glucose, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances, and the like that do not deleteriously react with the active compounds.

### EXAMPLES

### Example 1

Employing ethyl acetate (EA) solvent is used for formulating NPs This example describes methods that were employed using ethyl acetate solvent for formulating nanoparticles. Ethyl acetate is more acceptable solvent (regulatory point of view) compared to chloroform or methylene chloride (Class 2 solvents, as these are carcinogenic, chloroform concentration limit <60 PPM, methylene chloride (600 PPM) which is also used for making PLGA-based nanoparticles. The residual amount of EA (Class 3 solvent, that is allowed is 5,000 PPM (FDA guidelines, June 2017). However, EA is less volatile and hence is retained in the formulation, that affects the TFF process, particularly because it enlarges the pore size of TFF column membrane, leaching nanoparticles or rupturing the membrane the column. Hence the following protocol was developed to ensure removal of EA prior to TFF. The production process is divided into four major steps, each of these steps optimized to achieve NPs with antioxidant enzymes of specific characteristics. These steps are: 1) Reagent preparation, 2) Emulsification, 3) Formulation of NPs, and 4) Recovery of NPs and lyophilization. The protocol for antioxidant NP preparation 1 g batch using ethyl acetate as the solvent for PLGA was performed as follows:
1. Poly (dl-lactide co-glycolide), PLGA solution: PLGA (0.76-0.94 dL/g inherent viscosity; 50:50) is dissolved in 10 mL ethyl acetate by stirring overnight on a magnetic stirrer. In a typical 1 g batch, 810 mg PLGA+90 mg Dimethyl tartaric acid (DMT) are dissolved. Nanoparticles are also formulated with and without DMT.
2. Polyvinyl alcohol, PVA solution preparation—PVA—6 g is dissolved in 100 mL Mili Q water either by sonicated @RT for 7 min using Hielscher Sonicator (amplitude 70-80%) or by stirring overnight. The PVA solution is centrifuged at 4,000 rpm for 10 min to remove undissolved PVA there is any and then filtered using a 0.22-micron filter. Alternatively, PVA solution is centrifuged to remove undissolved PVA.
3. Human serum albumin (HSA—285 mg); recombinant human superoxide dismutase, SOD (5 mg) and recombinant human catalase, CAT (10 mg)—total 300 mg. All the three were added together to 2 mL MiliQ water in 5 mL Eppendorf tube and kept overnight in refrigerator (4 deg C) for dissolving.
4. Addition of HSA+SOD+CAT mixture to PLGA soln. Added in 5 to 7 portions; vortexed for 1 min and then sonicated using Q Sonica probe Sonicator for 3 min the vial kept in ice. Temperature is monitored and kept below 15° C. This forms water-in-oil (w/o) emulsion.
5. PVA solution (120 mL, 2%). The above 6% PVA stock solution is diluted so that the PVA concentration is 2% w/v. The PVA solution in a glass beaker and kept in a dry ice and allowed to cool (>10 deg C. but not freeze). Four mL—100% ethyl acetate was added to saturate the PVA solution. The above w/o emulsion is added to PVA solution in 4 to 5 small portions and sonicated for 1 min and a pause is given between each addition to maintain the temperature around 4 deg C. After adding of all the portions, sonicated for additional 2 min. Total sonication time is 6 min
6. The above emulsion was then passed through the homogenizer using EmulsiFlex at 10,000 psi, for ten cycles. The time required for one cycle to complete is first determined and then EmulsiFlex in run for the precalculated time. The steps #5 and #6 form water-in-oil-water (w/o/w) emulsion.
7. The above w/o/w emulsion was transferred to one arm conical flask (250 mL) and covered with 0.22 micron PVDF membrane. The flask with emulsion was kept in an ice bath and connected to a vacuum pump (vacuum—300 mmHg). The setup was left undisturbed for ˜4 hrs to evaporate ethyl acetate.
8. The emulsion was frozen using liquid nitrogen and lyophilized overnight to remove residual EA and then dispersed in 200 mL Mili Q water and sonicated for 3 min at 50% amplitude in Hielscher probe Sonicator and centrifuged at 4,000 rpm for 10 min at 4 deg C. and subjected to Tangential Flow Filtration (TFF—CD mode. TFF parameter—concentration 1 and diafiltration (DV—15 cycles). The PVA was analyzed before and after TFF to make sure >90% of PVA was washed out of the NP preparation.

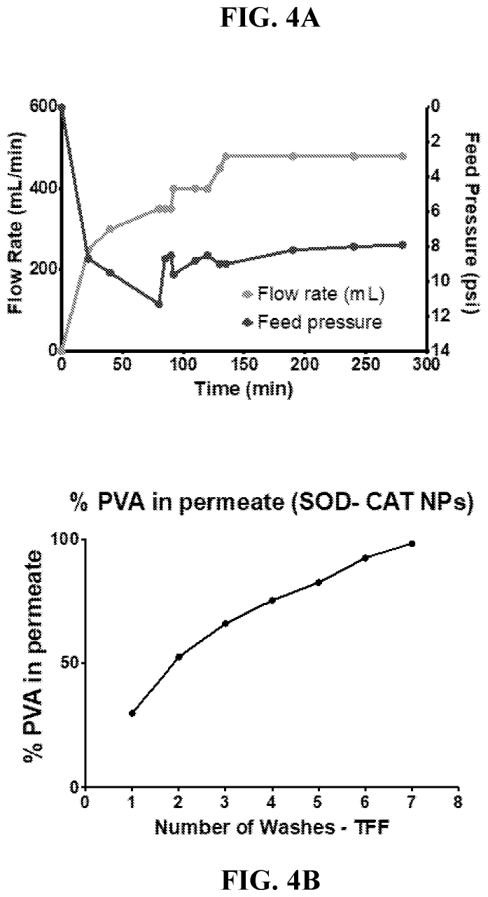

Then the formulation is sonicated (50% amplitude; Hielscher); and lyophilized for 2 days The parameters for tangential flow filtration (TFF) were monitored and optimized (FIG. 4A). As a part of the optimization of the TFF process, the number of washing cycles required to remove most of the PVA (used as an emulsifier) (FIG. 4B). The attempt was also to reduce the TFF processing time and make the process as automatic as possible.

TABLE 1
Optimization of Protocol at 1 g scale for control and bovine enzyme loaded Nanoparticles (nano-SOD/CAT)

| | Batch# | | | | | | | | | |
|---|---|---|---|---|---|---|---|---|---|---|
| | #1 | | #2 | | #3 (SOD/CAT | | #4 (SOD/CAT | | #5 (SOD/CAT | |
| | (Control) | | (Control) | | bovine) | | bovine) | | bovine) | |
| Batch Size | 1 | g | 1 | g | 1 | g | 1 | g | 1 | g |

Formulation Parameters

| Polymer/DMT | 810/90 | mg | 810/90 | mg | 810/90 | mg | 810/90 | mg | 810/90 | mg |
|---|---|---|---|---|---|---|---|---|---|---|
| Polymer Viscosity | 0.76-0.94 | | 0.76-0.94 | | 0.76-0.94 | | 0.76-0.94 | | 0.76-0.94 | |
| Composition | 50:50 | | 50:50 | | 50:50 | | 50:50 | | 50:50 | |
| (lactide to | | | | | | | | | | |
| glycolide ratio) | | | | | | | | | | |
| Human Albumin | 300 | mg | 300 | mg | 205 | mg | 205 | mg | 205 | mg |
| SOD | 0 | | 0 | | 40 | mg | 40 | mg | 40 | mg |
| CAT | 0 | | 0 | | 55 | mg | 55 | mg | 55 | mg |
| Homogenization | 15 | min | 15 | min | 20 | min | 20 | min | 20 | min |
| Evaporation | 4-6 | hrs | 4-6 | hrs | 4-6 | hrs | 4-6 | hrs | 4-6 | hrs |
| Lyophilization | 48 | hrs | 48 | hrs | 48 | hrs | 48 | hrs | 48 | hrs |

TFF (Tangential Flow Filtration)

| Column for TFF | D02-S05U-05-N | | D02-S05U-05-N | | D02-S05U-05-N | | D02-S05U-05-N | | D02-S05U-05-N | |
|---|---|---|---|---|---|---|---|---|---|---|
| (Repligen) | | | | | | | | | | |
| Flow Speed | 70-200 | ml/min | | | 50-200 | ml/min | 100-250 | ml/min | 80-200 | ml/min |
| Glucose | 10% | | 10% | | 10% | | 10% | | 10% | |
| Second Lyophilization | 48 | Hrs | 48 | Hrs | 48 | Hrs | 48 | Hrs | 48 | Hrs |

Characterization

| Hydrodynamic diameter | 231 | nm | 283 | nm | 227 | nm | 209 | nm |
|---|---|---|---|---|---|---|---|---|
| Polydispersity | 0.101 | | 0.062 | | 0.104 | | 0.033 | |
| Zeta potential | −7.85 | mv | −14.66 | mV | −8.29 | mV | −10.61 | mV |

| | | | | |
|---|---|---|---|---|
| Yield | 87% | 65% | 57% | 77% |

TABLE 2
Formulation of nanoparticles with human recombinant enzymes.

| | Batch# | | | | | |
|---|---|---|---|---|---|---|
| | #1 Human SOD | | #2 Human SOD | | #3 Human SOD | |
| | and CAT | | and CAT) | | and CAT) | |
| Batch Size | 1 | g | 1 | g | 250 mg | |
| | | | | | (3 times dose of | |
| | | | | | SOD and CAT) | |
| Polymer/DMT | 810/90 | mg | 810/90 | mg | 202/22 | mg |
| Polymer Viscosity | 0.76-0.94 | | 0.76-0.94 | | 0.76-0.94 | |
| Composition | 50:50 | | 50:50 | | 50:50 | |
| (lactide to | | | | | | |
| glycolide ratio) | | | | | | |
| Human Albumin | 285 | mg | 285 | mg | 60.8 | mg |
| SOD (Human) | 5 | mg | 5 | mg | 4 | mg |
| CAT (Human) | 10 | mg | 10 | mg | 12 | mg |
| Homogenization | 15 | min | 15 | min | 15 | min |
| Evaporation | 4-6 | hrs | 4.5 | hrs | 5 | hrs |
| Lyophilization | 48 | hrs | 48 | hrs | 48 | hrs |
| Column for TFF | D02-S05U-05-N | | D02-S05U-05-N | | D02-S05U-05-N | |
| (Repligen) | | | | | | |
| Flow Speed | 100-250 | ml/min | 250-350 | ml/min | 250 | ml/min |
| Glucose | 90% | | 90% | | 10% | |
| Second | 48 | Hrs | 48 | Hrs | 48 | Hrs |
| Lyophilization | | | | | | |
| Mean Size | 246 | nm | 250 | nm | 173 | nm |
| Polydispersity | 0.1 | | 0.050 | | 0.099 | |
| Zeta potential | −9.47 | mV | −10.4 | mV | −12.29 | mV |

| | | | |
|---|---|---|---|
| Yield | 92% | 57% | 57% |

Modification of TFF process to improve recovery of nano-SOD/CAT: In an attempt to make the process of Tangential Flow Filtration (TFF) more efficient, particularly to shorten the time of recovery (from ˜8 hrs to 2 hrs) and to make the process automatic, we noticed lower yield of Pro-NPs. The product yield dropped from ˜80% to below 60% with the modified TFF protocol. Upon thorough review of the TTF process, it was noticed that nanoparticle formulation becomes highly concentrated towards the end stage; hence, we suspected that a fraction of nanoparticles might have been retained in the porous membrane of the TFF column. To dislodge these nanoparticles stuck into the membrane, we used reverse flow (flow of water from outside of column to inside) that resulted in the recovery of nanoparticles. We also optimized those three times reverse flow (˜5 min each) was found to recover most of the nanoparticles stuck in the column. This additional step has improved the yield of nanoparticles from 57% to ˜83% (Table 3). Using the optimized protocol, the formulations of nano-SOD/CAT were prepared for dose #1, and dose #2. Dose #2 contains 3 times the amount of recombinant enzymes as in dose #1.

TABLE 3
Effect of reverse flow on yield of nanoparticles. All the formulations of
nano-SOD-CAT were prepared with recombinate human enzymes at 1 g scale

| | Date of Batch Preparation | | | |
|---|---|---|---|---|
| | Aug. 29, 2019 | Oct. 22, 2019 | Oct. 28, 2019 | Dec. 4, 2019 |
| | (without | (with | (with | (with |
| | reverse flow) | reverse flow) | reverse flow) | reverse flow) |
| | Dose | | | |
| | Dose #1 | Dose #2 | Dose #1 | Dose #2 |
| Hydrodynamic | 259 | 236 | 254 | 277 |
| diameter (nm) | | | | |
| Polydispersity | 0.049 | 0.033 | 0.08 | 0.09 |
| Index | | | | |
| Zeta Potential | −10.4 | −12.8 | −16.9 | −17.2 |
| (mV) | | | | |
| Yield | 57 | 87 | 78 | 83 |

TABLE 4
Characteristics of nano-SOD/CAT prepared at
1 g scale with recombinant human enzymes

| Parameters | Characteristics | |
|---|---|---|
| Mean hydrodynamic diameter | 246 | nm |
| Polydispersity Index (PI) | 0.1 | |
| Zeta Potential | −9.47 | mV |
| Catalytic activity per mg | | |
| nano-SOD/CAT* | | |

| | |
|---|---|
| SOD | 4203 ± 363 |
| CAT | 3249 ± 48 |
| Yield of nano-SOD/CAT post- | 92% |
| lyophilization | |

*Activity of SOD and CAT was measured following incubation of nano-SOD/CAT in a buffer, the supernatant was analyzed for the enzymatic activities using the respective assay kits for SOD (Dojindo Molecular Technologies, Inc. Rockville, MD) and CAT (Sigma-Aldrich). The above batch was tested for USP specifications.

TABLE 5
USP specifications for parenteral products. The
samples were characterized at Nova Biologicals

| | Test | |
|---|---|---|
| Description | Results | Comments |
| USP<71> Sterility testing: | Passed | No issue with sample preparation |
| set up and preparation for | | |
| 1st sample | | |
| USP <71> Validation | Passed | There is no interference of NPs |
| | | on testing protocol |
| USP <71> each additional | Passed | No evidence of bacterial growth |
| sample with initial set-up | | at 14 days |
| USP <85> Bacterial Endotoxin | Passed | 0.5 EU/ml |
| test | | |
| USP <788> Particulate Matter | Passed | 32 particles/ml greater than |
| in Injections | | 10 μm (USP limit <3000 particles) |
| | | 94 particles/ml greater than |
| | | 25 μm (USP limit <300 particles) |
| USP <791> pH | Passed | pH 7.1 |

Scale-up process for the production of antioxidant NPs was established but the yield was coming low. The process of recovery of NPs by Tangential Flow Filtration (TTF) was made more efficient, particularly reducing the time of recovery from 8 hrs to 2 hrs and making the process automatic. The issue was addressed by using the reverse flow to recover the formulation stuck in the TTF column (Table 3). Using the optimized protocols, NPs with dose #1 and dose #2 (three times the enzymes as dose #1) were successfully produced (Table 2).

The stability of enzyme-loaded NPs at different storage conditions (−20° C., 4-8° C., RT, and at 37° C.). There was no significant change in catalytic activity or physical properties of nano-SOD/CAT nanoparticles (particularly PLGA/PVA Sod/Catalase nanoparticles) when tested until 6 months. The data indicated that NPs could be stored in a refrigerator for a prolonged period, and exposure to room temperature does not affect its property.

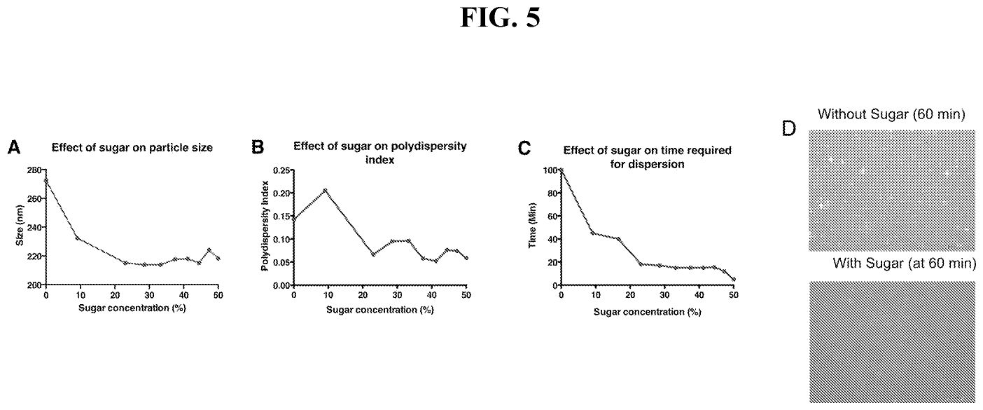

Optimization of cryoprotectant for easy dispersibility of antioxidant NPs. The amount of glucose to be added to NPs prior to lyophilization so that the formulation can be easily re-dispersed in saline for intravenous injection. The results show that beyond 20% glucose, there is no significant effect on particle size (FIG. 5A) or polydispersity index (FIG. 5B) but reduces the time required to redisperse nano-SOD/CAT. It takes only 5 min to redisperse when 1:1 w/w ratio -NPs and glucose were mixed prior to lyophilization (FIG. 5C). Under the microscope, the formulation with 50% glucose (1:1 w/w) looks quite monodispersed whereas the one with 10% glucose shows the presence of aggregates even at 60 min. Without the addition of any cryoprotect prior to lyophilization, the formulation requires sonication for redispersion, or it takes a long time (>100 min) to redisperse (C). Both these options are not practical in a clinical setting. With glucose added as a cryoprotect, the formulation of nano-SOD/CAT can be easily redispersed just by gentle mixing with saline. Under the microscope, nano-SOD/CAT without added sugar prior to lyophilization shows aggregates whereas with added sugar there was no aggregates (FIG. 5D).

Near-infrared dye-loaded NPs were prepared similarly to nano-SOD/CAT, but the dye was dissolved in the polymer solution (about 250 μg of NIR dye/100 mg PLGA) before emulsification. Near-infrared (NIR, SDB5700) dye was obtained from H.W. Sands Corp. (Jupiter, FL). The incorporated dye in the polymer phase acts as a marker for NPs and provides a quantitative signal proportional to the amount of NPs. Nano-SOD/CAT containing Latanoprost, a prostaglandin analogue was prepared by dissolving it in the PLGA polymer solution (1 mg/250 mg PLGA) prior to emulsification into PVA solution.

### Example 2

### Characterization of Nano-SOD/CAT

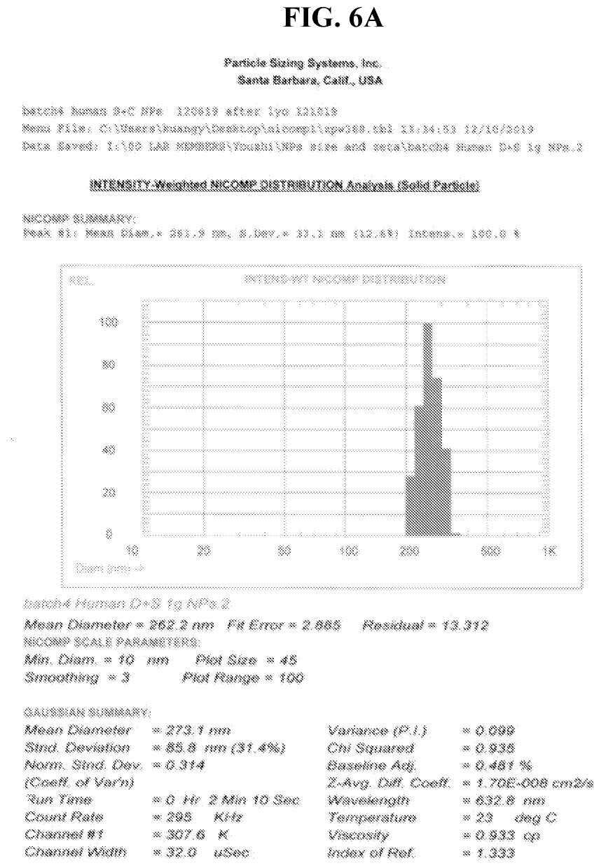

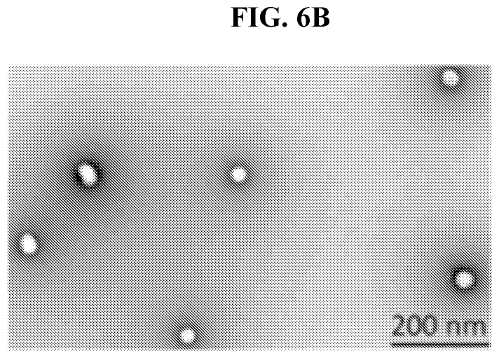

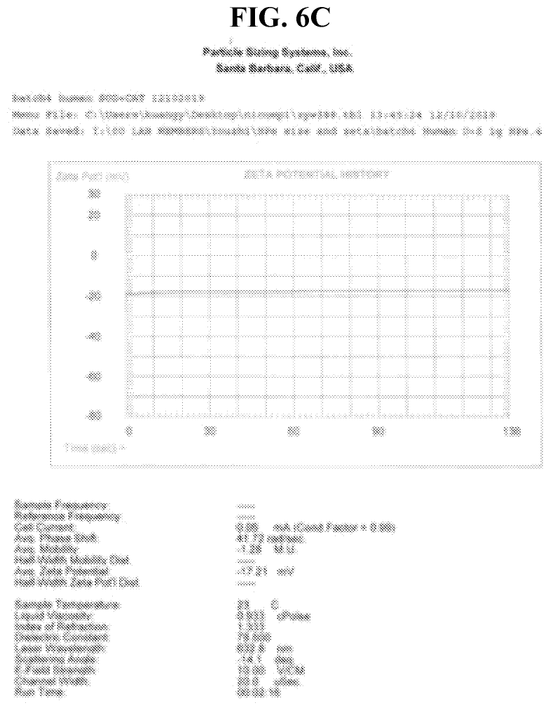

Hydrodynamic diameter and zeta potential of antioxidant NPs were determined using NICOMP 380 ZLS (Particle Sizing Systems, Port Richey, FL) (FIG. 6A). A stock dispersion of lyophilized antioxidant NPs was prepared in MQ water (2 mg/ml). Three μl of the above stock was diluted to 0.5 ml in MQ water for measuring particle size. The formulations were further characterized by transmission electron microscopy (TEM). For TEM, 5 μL of the NP-dispersion in water (˜100 μg/mL) was placed on a 200 mesh Formvar-coated TEM grid with a size of 97 μm (TED PELLA, Redding, CA). The samples were negatively stained with 2% w/v uranyl acetate solution, the excess stain was removed with filter paper, and the grid with coated NPs was washed with sterile MiliQ water and air-dried for 3 hours. The coated samples were observed with an electron microscope operating at 80 kV (Tecnai G2 SpiritBT, FEI Company, Hillsboro, OR) (FIG. 6B). A 100 μl of the stock was diluted to 3 ml in MQ water for measuring zeta potential. The size of NPs was measured at a scattering angle of 90° at 25° C. Zeta potential was measured in the phase-analysis mode and the current mode at a scattering angle of −14° (FIG. 6C). The encapsulation efficiency of each enzyme was determined from the difference in the amount added in the formulation and the amount seen in the supernatant and washings (unencapsulated). There is no interference of SOD on CAT assay and vice versa.

Determination of residual PVA: The amount of PVA associated with nanoparticles was determined by a colorimetric method based on forming a colored complex between adjacent hydroxyl groups of PVA and iodine molecule. About 2 to 5 mg of lyophilized nanoparticle sample was treated with 2 ml of 0.5 M NaOH for 15 min at 60 C. The sample was neutralized with 900 ml of 1 N HCl and the volume was adjusted to 5 ml with distilled water. To the sample, 3 ml of a 0.65 M solution of boric acid, 0.5 ml of a solution of I/KI (0.05 M/0.15 M), and 1.5 ml of distilled water were added. The absorbance of the samples was measured at 690 nm after 15 min incubation. A standard plot of PVA was prepared under identical conditions. The data were normalized to per mg nanoparticle weight. Using the above method, the residual PVA with the nano-SOD/CAT was 3.4% w/w.

### Example 3

The 5XFAD mice (Jackson Laboratory) model of AD was used as it demonstrates accelerated amyloid plaque deposition, gliosis, and progressive neuronal and synaptic loss accompanied by cognitive impairment, recapitulating several pathological hallmarks of human AD _{[24]}. This model shows amyloid-beta plaques at two months and cognitive impairment at three months; hence they are suitable for evaluating the treatment efficacy.

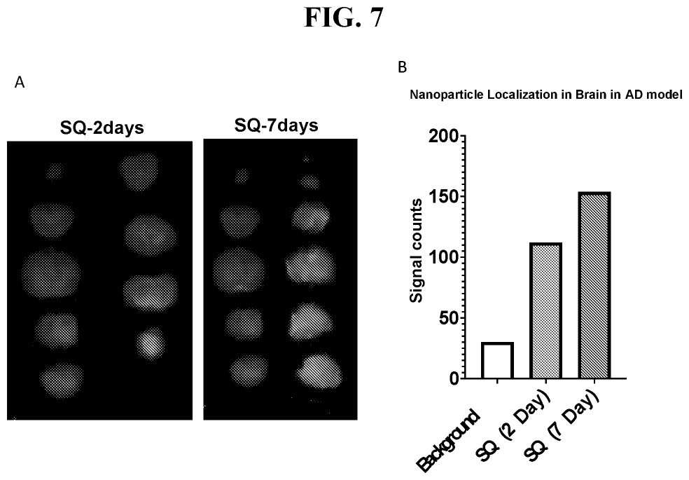

Uptake of nanoparticles from the SQ injection site to the brain in 5XFAD mice via the lymphatic route. The near-infrared dye-loaded nanoparticles were used to determine the uptake. The incorporated dye acts as a marker for nanoparticles. A single dose (60 mg/kg) of the dye-loaded nanoparticles suspended in saline was injected SQ. For this study, mice of 9-10 weeks were used because, by this age, they develop AD pathology in the brain. The animals were euthanized 2 and 7 days post nanoparticle administration. The animals were perfused with saline via cardiac puncture to remove blood. The harvested brains were cut into transverse sections (a total of 5 sections for each brain) using. The brain sections were imaged using Maestro Optical System for signal due to nanoparticle localization in the brain. The images were compared to the brain sections of the animals that did not receive any nanoparticles (FIG. 7A). The signal intensity for each brain section was quantified, and the signal from all the sections was combined (FIG. 7B).

### Example 4

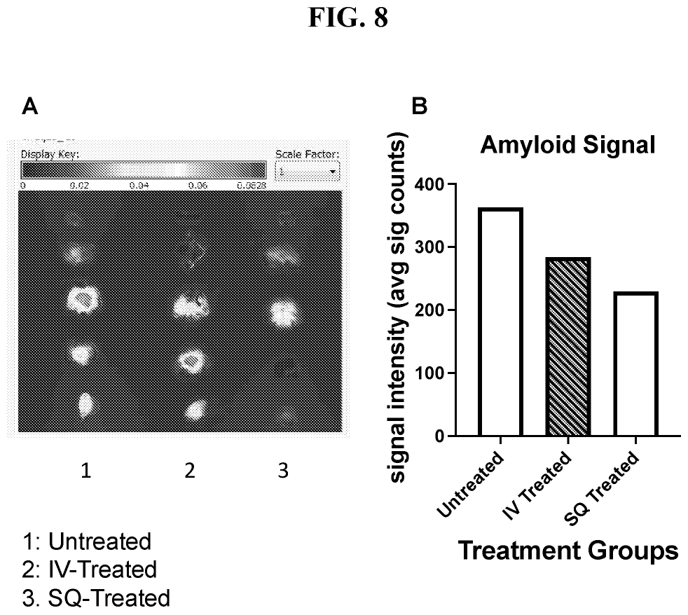

Evaluating the effect of treatment with antioxidant nanoparticles on inhibition of AD pathology. The treatment with antioxidant nanoparticles (nano-SOD/CAT) SQ was given to 5XFAD mice (60 mg/kg) at age of 5 weeks, every week for the first four weeks and thereafter once in two weeks. The treated animals were evaluated for amyloid β Maestro at the age of 16 weeks (about 12 weeks post-treatment), and the results were compared with untreated animals. Imaging and Quantification of Plaque Progression. The mice from the above treatment group were given an intraperitoneal (IP) injection of methoxy-X04 (5 mg/kg) (Tocris Bioscience, Cat. No. 4920) to label amyloid plaques. Methoxy-X04 is a fluorescent amyloid β (Aβ) probe for the detection and quantification of plaques, tangles, and cerebrovascular amyloid. Twenty-four hours after IP injection, mice are euthanized and perfused with 1×PBS. Intact brains were dissected into 5 transverse sections, and optical images of the sections were taken using Maestro EX Optical Imaging System (Caliper Life Sciences, Hopkinton, MA) and quantified (FIG. 8).

### Example 5

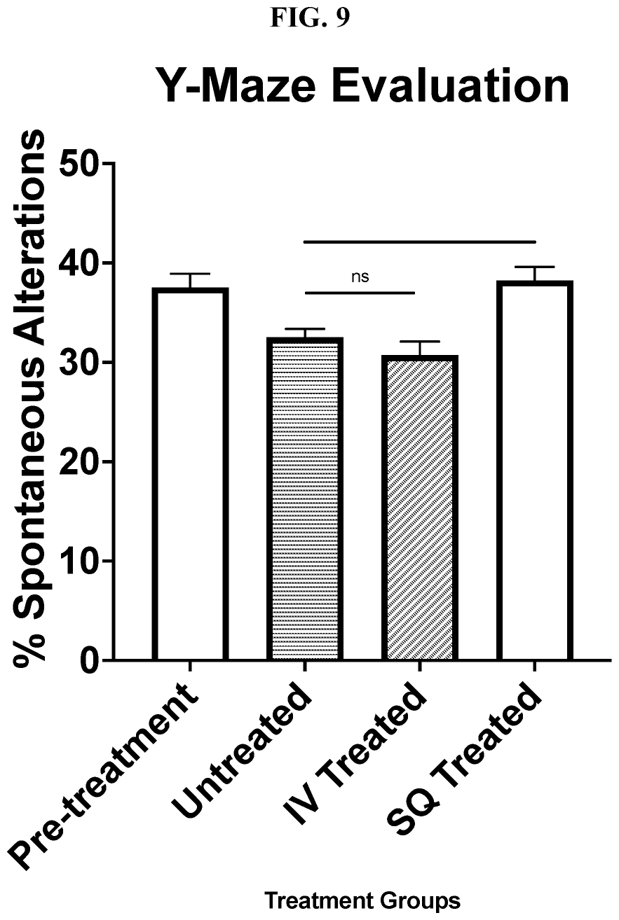

Evaluating the effect of treatment with antioxidant nanoparticles on behavior. The treatment with antioxidant nanoparticles (nano-SOD/CAT) was SQ given to 5XFAD mice (60 mg/kg) at age of 5 weeks, every week for the first four weeks and thereafter once in two weeks. The following parameters were used to determine the effect of the treatment. Y-Maze test: Y Maze or spontaneous alternation is a behavioral test for measuring short-term memory and assessing the willingness of rodents to explore novel environments (i.e., arms of the y-maze in a unique sequence). Mice typically prefer to investigate a new arm of the maze rather than return to one that was previously visited. The Y-shaped maze, with three identical arms (50×10×35 cm) positioned at equal angles (120°) was built with gray acrylic. Experimental mice will be placed at the end of one arm and allowed to move freely through the Y-maze. The start arm will be varied between mice to avoid placement bias. The series of arm entries are recorded visually, and an arm entry is considered to be complete when the hind paws of the mouse are fully placed in the arm. Alternations will be defined as successive entries into the three arms (ABC) on overlapping triplet sets (FIGS. 9A and B).

Novel object recognition test: Novel object recognition is a highly validated test for recognition memory. In this test, the animals are exposed to two or more objects and get to explore these for a while. Then, one of the objects is replaced by another object. If the subject's memory functions normally, they spend more time exploring novel objects than exploring familiar object(s). If the exploration of all objects is the same, this behavior can be interpreted as a memory deficit (FIG. 9C).

### Example 6

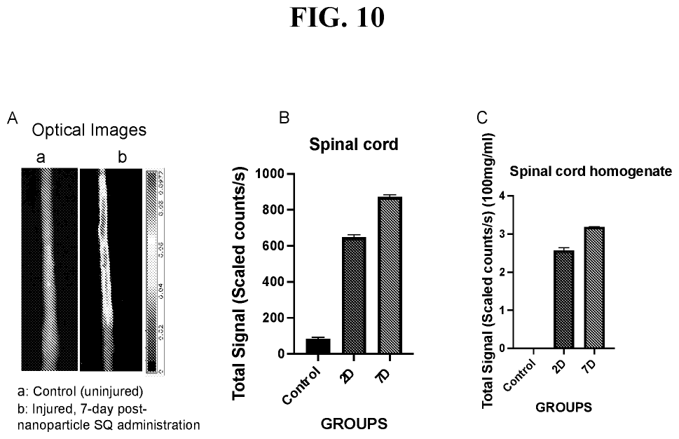

Localization of nanoparticles at the lesion site in spinal cord injury model following SQ injection. Near infrared-dye-loaded nanoparticles were injected SQ (30 mg/kg) to the rats with spinal cord injury. The spinal cords were harvested at 2 days and 7 days post-administration, and the lesion sites were imaged using Maestro optical imaging system for optical signal images (10A), the signal count (FIG. 10B). Following imaging of the spinal cord, the lesion sites were homogenized; homogenates were loaded into 96-well plated and imaged for the total signal count (FIG. 10C).

### Example 7

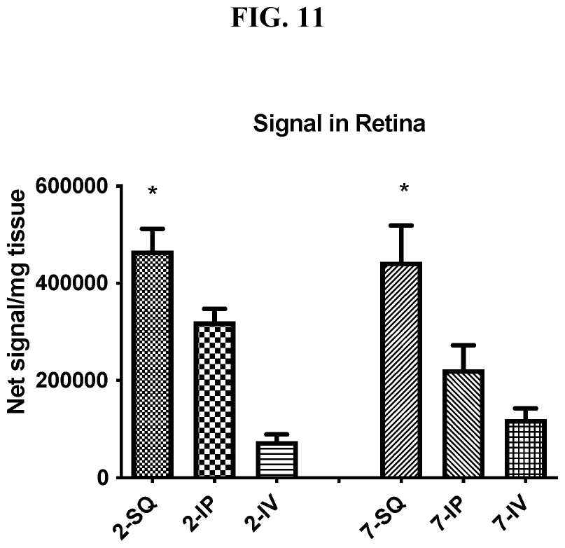

Localization of nanoparticles in the retina. The rd10 mouse model of retinitis pigmentosa was used. The animals were treated with near-infrared dye-loaded nanoparticles (120 mg/kg), and the retinas were harvested for imaging. The other groups included intravenous (IV) and Intraperitoneal injection (IP) of dye-loaded nanoparticles for comparison (FIG. 11).

### Example 8

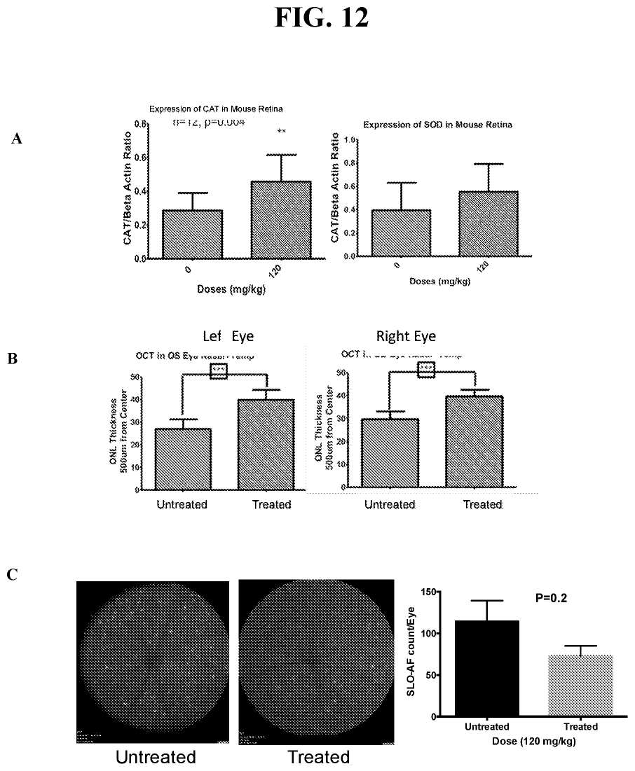

To analyze retinal tissue antioxidant enzyme levels in nano-SOD/CAT treated (120 mg/kg) vs. untreated group in rd10 mouse model of RP. Following intraperitoneal dosing of nano-SOD/CAT at age, P6, P12, and P18 in rd10 mouse model of RP and retinal analyses conducted at age P21. Retinal tissue for catalase (CAT) and SOD levels using western blot. The data showed significantly higher CAT activity in the treated group vs. in the untreated control, whereas SOD levels showed a trend towards increased activity in the treated group (FIG. 12A).

Following the treatment as above, to analyze retinal tissue for outer nuclear layer (ONL) thickness using optical coherence tomography (OCT) was used. The data show significantly greater ONL thickness in the treated group vs. in the untreated control, thus providing direct evidence of the protective efficacy of nano-SOD/CAT treatment on inhibiting retinal degeneration (FIG. 12B). Monocytes, macrophages, and microglia (MMM) play important roles in the retina by clearing cellular debris. RP is a retinal degenerative disease and hence shows MMM migration. The data show a lesser number of white spots, considered to be MMM, in the treated group as compared to in untreated control (FIG. 12C)), thus suggesting reduced retinal cell degeneration, which directly supports greater ONL thickness in the treated group (FIG. 12B).

### REFERENCES

1. Ashok, A.; Andrabi, S. S.; Mansoor, S.; Kuang, Y.; Kwon, B. K.; Labhasetwar, V. Antioxidant therapy in oxidative stress-induced neurodegen-erative diseases: Role of nanoparticle-based drug delivery sys-tems in clinical translation. Antioxidants 2022, In revision.
2. Tripathy, K.; Geetha, R. Latanoprost. In StatPearls; StatPearls Publishing Copyright © 2022, StatPearls Publishing LLC.: Treasure Island (FL), 2022.
3. Tam, A. L.; Gupta, N.; Zhang, Z.; Yicel, Y. H. Latanoprost Stimulates Ocular Lymphatic Drainage: An In Vivo Nanotracer Study. Transl Vis Sci Technol 2013, 2, 3, doi:10.1167/tvst.2.5.3.
4. Jacob, L.; de Brito Neto, J.; Lenck, S.; Corcy, C.; Benbelkacem, F.; Geraldo, L. H.; Xu, Y.; Thomas, J. M.; El Kamouh, M. R.; Spajer, M.; et al. Conserved meningeal lymphatic drainage circuits in mice and humans. J Exp Med 2022, 219, doi:10.1084/jem.20220035.
5. Karran, E.; De Strooper, B. The amyloid hypothesis in Alzheimer disease: new insights from new therapeutics. Nat Rev Drug Discov 2022, 21, 306-318, doi:10.1038/s41573-022-00391-w.
6. Rusu, M. C.; Nicolescu, M. I.; Vrapciu, A. D. Evidence of lymphatics in the rat eye retina. Ann Anat 2022, 244, 151987, doi:10.1016/j.aanat.2022.151987.
7. Clahsen, T.; Hadrian, K.; Notara, M.; Schlereth, S. L.; Howaldt, A.; Prokosch, V.; Volatier, T.; Hos, D.; Schroedl, F.; Kaser-Eichberger, A.; et al. The novel role of lymphatic vessels in the pathogenesis of ocular diseases. Prog Retin Eye Res 2023, 101157, doi:10.1016/j.preteyeres.2022.101157.
8. Uddin, N.; Rutar, M. Ocular Lymphatic and Glymphatic Systems: Implications for Retinal Health and Disease. Int J Mol Sci 2022, 23, doi:10.3390/ijms231710139.
9. Breazzano, M. P.; Grewal, M. R.; Tsang, S. H.; Chen, R. W. S. Etiology of Retinitis Pigmentosa. Methods Mol Biol 2023, 2560, 15-30, doi:10.1007/978-1-0716-2651-1_2.
10. Vingolo, E. M.; Casillo, L.; Contento, L.; Toja, F.; Florido, A. Retinitis Pigmentosa (RP): The Role of Oxidative Stress in the Degenerative Process Progression. Biomedicines 2022, 10, doi:10.3390/biomedicines10030582.
11. Kanan, Y.; Hackett, S. F.; Taneja, K.; Khan, M.; Campochiaro, P. A. Oxidative stress-induced alterations in retinal glucose metabolism in Retinitis Pigmentosa. Free Radic Biol Med 2022, 181, 143-153, doi:10.1016/j.freeradbiomed.2022.01.032.
12. Akbar, M.; Essa, M. M.; Daradkeh, G.; Abdelmegeed, M. A.; Choi, Y.; Mahmood, L.; Song, B. J. Mitochondrial dysfunction and cell death in neurodegenerative diseases through nitroxidative stress. Brain Res 2016, 1637, 34-55, doi:10.1016/j.brainres.2016.02.016.
13. Wojsiat, J.; Zoltowska, K. M.; Laskowska-Kaszub, K.; Wojda, U. Oxidant/Antioxidant Imbalance in Alzheimer's Disease: Therapeutic and Diagnostic Prospects. Oxid Med Cell Longev 2018, 2018, 6435861, doi:10.1155/2018/6435861.
14. Li, A.; Yue, L.; Xiao, S.; Liu, M. Cognitive Function Assessment and Prediction for Subjective Cognitive Decline and Mild Cognitive Impairment. Brain Imaging Behav 2022, 16, 645-658, doi:10.1007/s11682-021-00545-1.
15. Merighi, S.; Nigro, M.; Travagli, A.; Gessi, S. Microglia and Alzheimer's Disease. Int J Mol Sci 2022, 23, doi:10.3390/ijms232112990.
16. Louveau, A.; Smirnov, I.; Keyes, T. J.; Eccles, J. D.; Rouhani, S. J.; Peske, J. D.; Derecki, N. C.; Castle, D.; Mandell, J. W.; Lee, K. S.; et al. Structural and functional features of central nervous system lymphatic vessels. Nature 2015, 523, 337-341, doi:10.1038/nature14432.
17. Papadopoulos, Z.; Herz, J.; Kipnis, J. Meningeal Lymphatics: From Anatomy to Central Nervous System Immune Surveillance. J Immunol 2020, 204, 286-293, doi:10.4049/jimmunol.1900838.
18. Skobe, M.; Detmar, M. Structure, function, and molecular control of the skin lymphatic system. J Investig Dermatol Symp Proc 2000, 5, 14-19, doi:10.1046/j.1087-0024.2000.00001.x.
19. Bach, C.; Lewis, G. P. Lymph flow and lymph protein concentration in the skin and muscle of the rabbit hind limb. J Physiol 1973, 235, 477-492.
20. Zhang, X. Y.; Lu, W. Y. Recent advances in lymphatic targeted drug delivery system for tumor metastasis. Cancer Biol Med 2014, 11, 247-254, doi:10.7497/j.issn.2095-3941.2014.04.003.
21. Oshiro, H. The role of the lymphatic system in rabbit models for cancer metastasis research: a perspective from comparative anatomy. Okajimas Folia Anat Jpn 2014, 91, 25-28.
22. Richter, W. F.; Bhansali, S. G.; Morris, M. E. Mechanistic determinants of biotherapeutics absorption following SC administration. AAPS J 2012, 14, 559-570, doi:10.1208/s12248-012-9367-0.
23. Nair, A. B.; Jacob, S. A simple practice guide for dose conversion between animals and human. Journal of basic and clinical pharmacy 2016, 7, 27-31, doi:10.4103/0976-0105.177703.
24. Oakley, H.; Cole, S. L.; Logan, S.; Maus, E.; Shao, P.; Craft, J.; Guillozet-Bongaarts, A.; Ohno, M.; Disterhoft, J.; Van Eldik, L.; et al. Intraneuronal beta-amyloid aggregates, neurodegeneration, and neuron loss in transgenic mice with five familial Alzheimer's disease mutations: potential factors in amyloid plaque formation. J Neurosci 2006, 26, 10129-10140, doi:10.1523/jneurosci.1202-06.2006.
   All of the above references are herein incorporated by reference.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of treating a neurodegenerative condition and/or neurological condition and/or an eye condition with nanoparticles comprising:
administering a composition comprising a plurality of nanoparticles to a subject via subcutaneous, intraperitoneal, or intramuscular injection such that said plurality of nanoparticles are taken up by lymphatic capillaries and are transported to neurological tissue and/or eye tissue of said subject via said subject's lymphatic system,
wherein said subject has a neurodegenerative condition and/or neurological condition and/or eye condition and
wherein said plurality of nanoparticles encapsulate, or are attached or absorbed to, at least one drug agent.

2. The method of claim 1, wherein said nanoparticles: i) have an average diameter of about 10-115 nm, about 20-100 nm, or about 60-90 nm as measured by transmission electron microscopy (TEM), or a hydrodynamic diameter of 3-10 nm, 30 to 340 nm, or about 200-250 nm, and/or ii) optionally have a zeta potential of −10 mV to −20 mV or 0 to −30 mV; and/or
wherein said at least one drug agent is selected from the group consisting of: a prostaglandin analogue, vascular endothelial growth factor, magnesium, an anti-inflammatory drug, acetaminophen, an antioxidant enzyme, a neurotrophic factor, and an angiogenic factor.

3. The method of claim 1, wherein said at least one drug agent comprises one, or combinations of, the following:
i) an antioxidant enzyme, which is optionally: superoxide dismutase (SOD), a catalase; a peroxiredoxin (PRX), a glutathione peroxidase (GPX), ascorbate peroxidase, monodehydroascorbate reductase, dehydroascorbate reductase, and glutathione reductase;
ii) an anti-inflammatory drug, which is optionally: ibuprofen, naproxen, diclofenac, celecoxib, mefenamic acid, etoricoxib, indomethacin, aspirin, or a steroid;
iii) an angiogenic factor, which is optionally: a VEGF family molecule, a FGF family molecule, or angiopoietin;
iv) a neurotrophic factor, which is optionally: epidermal growth factor, basic fibroblast growth factor, brain-derived neurotrophic factor, and glial cell line-derived neurotrophic factor;
v) a prostaglandin or prostaglandin analogue, which is optionally:
A) a Prostaglandin F2α, which is optionally: Xalatan (latanoprost), Zioptan (tafluprost), Travatan Z (travoprost), Lumigan (bimatoprost), or Vyzulta (latanoprostene bunod);
B) a Prostaglandin E1, which is optionally Muse (alprostadil), Edex (alprostadil), Caverject (alprostadil), Caverject Impulse (alprostadil), or Cytotec (misoprostol);
C) a Prostaglandin E2, which is optionally: Cervidil (dinoprostone), or Prepidil (dinoprostone),
D) a Prostacyclin, which is optionally: Veletri (epoprostenol), Flolan (epoprostenol), Remodulin (treprostinil), Tyvaso (treprostinil), and Ventavis (iloprost);
E) a Progestin that dilates lymphatic vessels; and
vi) an Alzheimer's disease drug which is optionally: Aducanumab, Lecanemab, a Cholinesterase inhibitor, Donepezil, Rivastigmine, Galantamine, a Glutamate regulator, Memantine, combination of Donepezil and Memantine, Orexin receptor antagonist, and Suvorexant, inhibitor of BACE1.

4. The method of claim 1, wherein said subject has said eye condition and said administration is conducted in or near a lymphatic vessel on said subject's face, and wherein said eye condition is optionally selected from: a Corneal transplant, Dry eye, Herpes Simplex Virus (HSV-1), keratitis, Glaucoma, an Intraocular tumor, Uveitis, and Retinitis pigmentosa.

5. The method of claim 1, wherein said subcutaneous injection is performed at a location on the subject in the vicinity of deep cervical lymph nodes or lymph vessels thereof, such that said plurality of nanoparticles are taken up by said lymphatic capillaries and are transported to said neurological tissue.

6. The method of claim 5, wherein said deep cervical lymph nodes are selected from: Anterior Cervical Lymph Nodes, Deep Cervical Lymph Nodes, and Inferior Deep Cervical Lymph Nodes.

7. The method of claim 6, wherein said subcutaneous injection is performed at a location on the subject within about 15 mm, 12 mm, 10 mm, 8 mm 5 mm or 3 mm of said deep cervical lymph nodes or lymph vessels thereof, such that said plurality of nanoparticles are taken up by lymphatic capillaries and are transported to neurological tissue.

8. The method of claim 1, wherein said subcutaneous injection is performed at a location on the subject in the vicinity of the inner canthus lymph vessel, the outer canthus lymph vessel, or the inferior eyelid lymph vessel, such that said plurality of nanoparticles are taken up by lymphatic capillaries and are transported to eye tissue.

9. The method of claim 8, wherein said subcutaneous injection is performed at a location of the subject within about 15 mm, 12 mm, 10 mm, 8 mm 5 mm, or 3 mm of said inner canthus lymph vessel, said outer canthus lymph vessel, or said inferior eyelid lymph vessel, such that said plurality of nanoparticles are taken up by lymphatic capillaries and are transported to eye tissue.

10. The method of claim 1, wherein said subcutaneous or intraperitoneal or intramuscular injection is performed at a location within about 15 mm, 12 mm, 10 mm, 8 mm 5 mm, or 3 mm on the subject in the vicinity of lymph vessels or lymph nodes, such that said plurality of nanoparticles are taken up by lymphatic capillaries and are transported to said neurological tissue or said eye tissue.

11. The method of claim 1, wherein said administering is via subcutaneous injection, and wherein the subcutaneous site of administration on the subject is warmed and/or massaged before, during, or after said administering.

12. The method of claim 1, wherein said nanoparticles have at least one of the following properties: i) a hydrodynamic diameter range of 100 to 300 nm, or about 262 nm; ii) a transmission electron microscopic (TEM) diameter of about 10-115 nm, about 20-100 nm, about 60-90 nm as measured by transmission electron microscopy (TEM); a hydrodynamic diameter of 30 to <350 nm or about 200-250 nm, and zeta potential −10 mV to −20 mV or 0 to −30 mV.

13. The method of claim 1, wherein said composition has a viscosity of about 1.0 to 10 centipoise (cps).

14. The method of claim 1, wherein said nanoparticles are present in said composition at a concentration of about 1 to 15 mg/ml, or about 10 mg/ml.

15. The method of claim 1, wherein said composition is administered at an infusion rate of about 20 to 80 ml per hour, and/or wherein said subject is a human, and/or said administering is conducted for about 20-40 minutes.

16. The method of claim 1, wherein said nanoparticles comprise a material selected from: a polymer, metal, dendrimers, gold, lipids, ceramic, inorganic-based nanomaterial, carbon-based nanomaterial, organic-based nanomaterial, and composite-based nanomaterial.

17. The method of claim 1, wherein said subject is a human, and/or said administering provides about 450 mg of said nanoparticles to said subject.

18. The method of claim 1, wherein said nanoparticles comprise poly (D,L-lactide co-glycolide) (PLGA) and/or polyvinyl alcohol (PVA), and optionally wherein said nanoparticles are composed of about 2-5% of said PVA, and optionally wherein said nanoparticles have a hydrodynamic diameter of 3-30 nm or 3-25 nm.

19. The method of claim 1, wherein said neurodegenerative and/or neurological condition is selected from the group consisting of: Parkinson's Disease, Alzheimer's Disease, Multiple Sclerosis (MS), Huntington's disease, and Amyotrophic Lateral Sclerosis (ALS).

20. The method of claim 1, wherein said neurological condition is selected from the group consisting of: traumatic brain injury, ischemia/reperfusion injury, a spinal injury, peripheral nerve injury, and a stroke, brain tumor, epilepsy, neural infection, and meningitis.

21. The method of claim 1, wherein said drug agent comprises SOD and catalase and optionally said SOD and said catalase are encapsulated, attached or absorbed to, separate nanoparticles.

22. The method of claim 21, wherein said SOD and said catalase are administered simultaneously to said subject.

23. The method of claim 1, further comprising: an additional administering wherein a plurality of said type of nanoparticles are administered intravenously to said subject before, during, or after the initial administering.

24. The method of claim 1, wherein said at least one drug agent comprises a prostaglandin or a prostaglandin analogue and is administered first, and wherein the method further comprises: administering additional nanoparticles that encapsulate, or are attached or absorbed to, one or more antioxidant enzymes which are optionally SOD and/or catalase.

25. The method of claim 1, wherein the nanoparticles are biodegradable.

26. The method of claim 1, wherein said administering is via continuous infusion over a time period of at least 1 minutes, 5 minutes, 30 minutes, 1 hour, or two hours.

27. The method of claim 1, wherein each mg of nanoparticles comprise SOD and is loaded with about 10 μg to about 150 μg SOD; and/or wherein each mg of nanoparticles comprise catalase and is loaded with about 10 μg to about 150 μg catalase.

28. The method of claim 1, further comprising: administering an additional agent that can dissolve or bind amyloid precursor protein (APP), amyloid plaques, and/or an agent to clear the brain lymphatic system to facilitate drainage of metabolic waste from the brain; and optionally wherein such agent is vascular endothelial growth factor.

29. The method of claim 1, wherein said drug agent comprise SOD and/or, and wherein after said administering, some of the SOD and the catalase is released rapidly from the one or more nanoparticles in the neurological tissue, and then the SOD and the catalase that remains in the one or more nanoparticles is released slowly over a sustained period of time in the neurological tissue.

30. The method of claim 1, further comprising administering one or more cells, growth factors, tissue grafts, antioxidants, hormones, steroids, vitamins, minerals or a combination thereof.

31. The method of claim 1, wherein said subject has a spinal cord injury, which is optionally a recent spinal cord injury.

32. The method of claim 1, wherein said subject is a human and/or wherein said composition further comprises about 15-60% glucose or about 20-50% glucose.

33. The method of claim 1, wherein said administering is performed by a paramedic.

34. The method of claim 1, wherein said neurological tissue comprises said subject's brain.

35. The method of claim 1, wherein said subject is a human, and wherein about 2.4 to 9.7 mg/kg of said nanoparticles are administered to said subject.

36. A system comprising:
a) an infusion pump or osmotic pump, which is optionally implanted in a subject; and
b) a composition comprising the nanoparticles described in any of claims 1-35, and optionally wherein said nanoparticles are located inside said infusion or osmotic pump.

37. The system of claim 36, wherein aid nanoparticles: i) have an average diameter of about 10-115 nm, about 20-100 nm, or about 60-90 nm as measured by transmission electron microscopy (TEM), or a hydrodynamic diameter of 30 to 340 nm, or about 200-250 nm, and/or ii) wherein said nanoparticles optionally have a zeta potential of −10 mV to −20 mV or 0 to −30 mV.

38. The system of claim 36, wherein said nanoparticles have at least one of the following properties: i) a hydrodynamic diameter range of i) about 10-115 nm or about 20-100 nm, about 60-90 nm as measured by transmission electron microscopy (TEM); ii) hydrodynamic diameter 30 to <350 nm, about 200-250 nm, and iii) a zeta potential −10 mV to −20 mV or 0 to −30 mV.

39. The system of claim 36, wherein said composition has a viscosity of about 1.0 to 10 centipoise (cps) and/or wherein said composition further comprises about 15-60% glucose or about 20-50% glucose.

40. The system of claim 36, wherein said nanoparticles are present in said composition at a concentration of about 1 to 15 mg/ml, or about 10 mg/ml; and/or wherein said nanoparticles comprise poly (D,L-lactide co-glycolide) (PLGA) and/or polyvinyl alcohol (PVA), and optionally wherein said nanoparticles are composed of about 2-5% of said PVA, and optionally wherein said nanoparticles have a hydrodynamic diameter of 3-30 nm or 3-25 nm.

41. A composition, system, or kit comprising:
a) a plurality of nanoparticles comprising poly (D,L-lactide co-glycolide) (PLGA), and/or polyvinyl alcohol (PVA), wherein optionally at least part of said PVA is associated with the nanoparticle surface, wherein said nanoparticles encapsulate, or are attached or absorbed to, at least drug agent; and
b) a cryoprotectant, which is optionally glucose.

42. The composition, system, or kit of claim 41, wherein said plurality of nanoparticles and glucose are combined in a mixed-composition.

43. The composition, system, or kit of claim 42, wherein said cryoprotectant, and said plurality of nanoparticles are present in said mixed-composition at about a 1:1 w/w ratio.

44. The composition, system, or kit of claim 42, wherein said cryoprotectant, and said plurality of nanoparticles are present in said mixed-composition at about a 0.4:1-1:0.4 w/w ratio; and/or wherein said nanoparticles are composed of about 2-5% of said PVA, and optionally wherein said nanoparticles have a hydrodynamic diameter of 3-30 nm or 3-25 nm.

45. The composition of claim 41, further wherein further comprising saline solution and/or wherein said cryoprotectant is present at about 15-60% or about 20-50% or 50-60% of said composition.

46. The composition, system, or kit of claim 41, wherein said at least one drug agent comprises one, or combinations of, the following:
i) an antioxidant enzyme, which is optionally: a superoxide dismutase (SOD), a catalase; a peroxiredoxin (PRX), a glutathione peroxidase (GPX), ascorbate peroxidase, monodehydroascorbate reductase, dehydroascorbate reductase, and glutathione reductase;
ii) an anti-inflammatory drug, which is optionally: ibuprofen, naproxen, diclofenac, celecoxib, mefenamic acid, etoricoxib, indomethacin, aspirin, or a steroid;
iii) an angiogenic factor, which is optionally: a VEGF family molecule, a FGF family molecule, or angiopoietin;
iv) a neurotrophic factor, which is optionally: epidermal growth factor, basic fibroblast growth factor, brain-derived neurotrophic factor, and glial cell line-derived neurotrophic factor;
v) a prostaglandin or prostaglandin analogue, which is optionally:
A) a Prostaglandin F 2α, which is optionally: Xalatan (latanoprost), Zioptan (tafluprost), Travatan Z (travoprost), Lumigan (bimatoprost), or Vyzulta (latanoprostene bunod);
B) a Prostaglandin E1, which is optionally Muse (alprostadil), Edex (alprostadil), Caverject (alprostadil), Caverject Impulse (alprostadil), or Cytotec (misoprostol);
C) a Prostaglandin E2, which is optionally: Cervidil (dinoprostone), or Prepidil (dinoprostone),
D) a Prostacyclin, which is optionally: Veletri (epoprostenol), Flolan (epoprostenol), Remodulin (treprostinil), Tyvaso (treprostinil), and Ventavis (iloprost);
E) Progestin, a dilator of lymphatic vesicles; and
vi) an Alzheimer's disease drug which is optionally: Aducanumab, Lecanemab, a Cholinesterase inhibitor, Donepezil, Rivastigmine, Galantamine, a Glutamate regulator, Memantine, combination of Donepezil and memantine, Orexin receptor antagonist, and Suvorexant.

47. The composition, system, or kit of claim 41, wherein said nanoparticles have at least one of the following properties: i) about 10-115 nm or about 20-100 nm, about 60-90 nm as measured by transmission electron microscopy (TEM); ii) hydrodynamic diameter 3-30 nm, 30 to <350 nm, about 200-250 nm, and iii) a zeta potential −10 mV to −20 mV or 0 to −30 mV.

48. The composition, system, or kit of claim 41, wherein said composition has a viscosity of about 1.0 to 10 centipoise (cps).

49. The composition, system, or kit of claim 41, wherein said nanoparticles are present in said composition at a concentration of about 1 to 15 mg/ml, or about 10 mg/ml.

50. A composition in a lyophilized form comprising:
a) a plurality of nanoparticles comprising poly (D,L-lactide co-glycolide) (PLGA), and/or polyvinyl alcohol (PVA), wherein optionally at least part of said PVA is associated with the nanoparticle surface, wherein said nanoparticles encapsulate, or are attached or absorbed to, at least one drug agent; and
b) a cryoprotectant, which is optionally glucose.

51. The lyophilized composition of claim 50, wherein said cryoprotectant, and said plurality of nanoparticles are present in said composition at about a 1:1 w/w ratio.

52. The lyophilized composition claim 50, wherein said cryoprotectant, and said plurality of nanoparticles are present in said composition at about a 0.4:1-1:0.4 w/w ratio.

53. The lyophilized composition of claim 50, wherein said at least one drug agent comprises one, or combinations of, the following:
i) an antioxidant enzyme, which is optionally: a superoxide dismutase (SOD), a catalase; a peroxiredoxin (PRX), a glutathione peroxidase (GPX), ascorbate peroxidase, monodehydroascorbate reductase, dehydroascorbate reductase, and glutathione reductase;
ii) an anti-inflammatory drug, which is optionally: ibuprofen, naproxen, diclofenac, celecoxib, mefenamic acid, etoricoxib, indomethacin, aspirin, or a steroid;
iii) an angiogenic factor, which is optionally: a VEGF family molecule, a FGF family molecule, or angiopoietin;
iv) a neurotrophic factor, which is optionally: epidermal growth factor, basic fibroblast growth factor, brain-derived neurotrophic factor, and glial cell line-derived neurotrophic factor;
v) a prostaglandin or prostaglandin analogue, which is optionally:
A) a Prostaglandin F 2α, which is optionally: Xalatan (latanoprost), Zioptan (tafluprost), Travatan Z (travoprost), Lumigan (bimatoprost), or Vyzulta (latanoprostene bunod);
B) a Prostaglandin E1, which is optionally Muse (alprostadil), Edex (alprostadil), Caverject (alprostadil), Caverject Impulse (alprostadil), or Cytotec (misoprostol);
C) a Prostaglandin E2, which is optionally: Cervidil (dinoprostone), or Prepidil (dinoprostone), and
D) a Prostacyclin, which is optionally: Veletri (epoprostenol), Flolan (epoprostenol), Remodulin (treprostinil), Tyvaso (treprostinil), and Ventavis (iloprost); and
vi) an Alzheimer's disease drug which is optionally: Aducanumab, Lecanemab, a Cholinesterase inhibitor, Donepezil, Rivastigmine, Galantamine, a Glutamate regulator, Memantine, combination of Donepezil and memantine, Orexin receptor antagonist, and Suvorexant.

54. The lyophilized composition of claim 50, wherein said nanoparticles have at least one of the following properties: i) about 10-115 nm or about 20-100 nm, about 60-90 nm as measured by transmission electron microscopy (TEM); ii) hydrodynamic diameter 3-30 nm, 30 to <350 nm, about 200-250 nm, and iii) a zeta potential −10 mV to −20 mV or 0 to −30 mV.

55. The lyophilized composition of claim 50, wherein said composition has a viscosity of about 1.0 to 10 centipoise (cps) and/or wherein said lyophilized composition further comprises about 15-60% glucose or about 20-50% glucose.

56. The lyophilized composition of claim 50, wherein said nanoparticles are present in said composition at a concentration of about 1 to 15 mg/ml, or about 10 mg/ml, and/or wherein said nanoparticles are composed of about 2-5% of said PVA, and optionally wherein said nanoparticles have a hydrodynamic diameter of 3-30 nm or 3-25 nm.

57. The lyophilized composition of claim 50, wherein said nanoparticles comprise a material selected from: a polymer, metal, dendrimers, gold, lipids, ceramic, inorganic-based nanomaterial, carbon-based nanomaterial, organic-based nanomaterial, and composite-based nanomaterial.

58. A composition comprising: a plurality of nanoparticles which optionally comprise poly (D,L-lactide co-glycolide) (PLGA), and/or polyvinyl alcohol (PVA),
wherein said plurality of nanoparticles encapsulate, or are attached or absorbed to, at least one drug agent,
wherein said nanoparticles: i) optionally have an average diameter of about 10-115 nm, about 20-100 nm, or about 60-90 nm as measured by transmission electron microscopy (TEM), or a hydrodynamic diameter of 3-30 nm, 30 to 340 nm, or about 200-250 nm, and/or ii) optionally have a zeta potential of −10 mV to −20 mV or 0 to −30 mV;
wherein said at least one drug agent comprises one, or combinations of, the following:
i) an antioxidant enzyme, which is selected from: a peroxiredoxin (PRX), a glutathione peroxidase (GPX), ascorbate peroxidase, monodehydroascorbate reductase, dehydroascorbate reductase, and glutathione reductase;
ii) an anti-inflammatory drug, which is optionally: ibuprofen, naproxen,
diclofenac, celecoxib, mefenamic acid, etoricoxib, indomethacin, aspirin, or a steroid;
iii) an angiogenic factor, which is optionally: a VEGF family molecule, a FGF family molecule, or angiopoietin;
iv) a neurotrophic factor, which is optionally: epidermal growth factor, basic fibroblast growth factor, brain-derived neurotrophic factor, and glial cell line-derived neurotrophic factor;
v) a prostaglandin or prostaglandin analogue, which is optionally:
A) a Prostaglandin F 2α, which is optionally: Xalatan (latanoprost), Zioptan (tafluprost), Travatan Z (travoprost), Lumigan (bimatoprost), or Vyzulta (latanoprostene bunod);
B) a Prostaglandin E1, which is optionally Muse (alprostadil), Edex (alprostadil), Caverject (alprostadil), Caverject Impulse (alprostadil), or Cytotec (misoprostol);
C) a Prostaglandin E2, which is optionally: Cervidil (dinoprostone), or Prepidil (dinoprostone), and
D) a Prostacyclin, which is optionally: Veletri (epoprostenol), Flolan (epoprostenol), Remodulin (treprostinil), Tyvaso (treprostinil), and Ventavis (iloprost); and
vi) an Alzheimer's disease drug which is optionally: Aducanumab, Lecanemab, a Cholinesterase inhibitor, Donepezil, Rivastigmine, Galantamine, a Glutamate regulator, Memantine, combination of Donepezil and memantine, Orexin receptor antagonist, and Suvorexant.

59. The composition of claim 58, wherein said nanoparticles further encapsulate, or are attached or absorbed to, an additional agent selected from human serum albumin, superoxide dismutase (SOD) and/or a catalase, a pore forming agent, Dimethyl tartaric acid (DMT), human serum albumin as stabilizer, PVA, and glucose.

60. The composition of claim 58, wherein said nanoparticles comprise a material selected from: a polymer, metal, dendrimers, gold, lipids, ceramic, inorganic-based nanomaterial, carbon-based nanomaterial, organic-based nanomaterial, and composite-based nanomaterial, imaging agent.

61. The composition of claim 58, wherein said composition further comprises about 15-60% glucose or about 20-50% glucose, and/or wherein said nanoparticles are composed of about 2-5% of said PVA, and optionally wherein said nanoparticles have a hydrodynamic diameter of 3-30 nm or 3-25 nm.

62. A method comprising:
a) dissolve poly (dl-lactide co-glycolide) (PLGA) in ethyl acetate to generate a PLGA solution, and optionally dissolving dimethyl tartaric acid (DMT) in said PLGA solution;
b) dissolve polyvinyl alcohol (PVA) in water to generate a PVA solution, and optionally removing any undissolved PVA by centrifugation and/or filtration;
c) at least one of the following:
i) dissolve human serum albumin and at least one drug agent in water to generate a drug agent solution, and
ii) dissolve a drug agent in ethyl acetate to generate a drug agent solution;
d) combine said drug agent solution and said PLGA solution to generate a combined solution
e) vortexing or otherwise treating said combined mixture to generate a first water-in-oil emulsion;
f) diluting said PVA solution such that a 2-5% PVA w/v solution is generated;
g) cooling said 2-5% PVA w/v solution such that is between 1 and 10 degrees Celsius;
h) adding ethyl acetate to said 2-5% PVA w/v solution such that it is saturated;
i) combining said water-in-oil emulsion with said 2-5% PVA w/v solution to generate a combined solution;
f) sonicating said combined solution to generate a sonicated solution;
g) passing said sonicated solution through a homogenizer such that a water-in-oil-water (w/o/w) emulsion is generated;
h) treating said w/o/w emulsion to evaporate ethyl acetate therefrom to generate a treated w/o/w emulsion comprising nanoparticles;
i) freezing and lyophilizing said nanoparticles to remove residual ethyl acetate;
j) dispersing said treated nanoparticles in water and to generate a nanoparticles dispersion;
k) subjecting said nanoparticles dispersion to tangential flow filtration to remove excess PVA; and
l) lyophilizing nanoparticles following washing steps in TFF to generate a lyophilized nanoparticle powder.
